(19) United States
(12) Patent Application Publication
(10) Pub. No.: US 2026/0306630 A1
(43) Pub. Date: Oct. 8, 2026

(54) METHODS AND MATERIALS FOR TREATING CANCER

(71) Applicant: Mayo Foundation for Medical Education and Research, Rochester, MN (US)

(72) Inventors: Richard G. Vile, Rochester, MN (US); Rosa M. Diaz Marcano, Rochester, MN (US); Timothy J. Kottke, Oronoco, MN (US); Jason M. Tonne, Rochester, MN (US)

(21) Appl. No.: 19/480,897

(22) PCT Filed: May 1, 2024
(86) PCT No.: PCT/US2024/027182

§ 371 Date: Nov. 3, 2025

Related U.S. Application Data
(60) Provisional application No. 63/463,817, filed on May 3, 2023.

(51) Int. Cl. A61K 40/24; A61K 40/19; A61K 48/00; A61P 35/00

(52) U.S. Cl. CPC A61K 40/24; A61K 40/19; A61K 48/0033; A61K 48/005; A61P 35/00

(57) ABSTRACT

This document relates to methods and materials involved in treating cancer. For example, methods and materials for using allogenic APCs (e.g., allogenic dendritic cells) designed to release a viral vector that can infect a T cell (e.g., an infectious retroviral vector or an infectious lentiviral vector) and drive expression of an antigen receptor (e.g., a CAR) within that T cell to produce dual specific CAR⁺ memory T cells and/or to treat cancer are provided.

### CROSS-REFERENCE TO RELATED APPLICATIONS

This application claims the benefit of U.S. Patent Application Ser. No. 63/463,817, filed on May 3, 2023. The disclosure of the prior application is considered part of, and is incorporated by reference in, the disclosure of this application.

### SEQUENCE LISTING

This application contains a Sequence Listing that has been submitted electronically as an XML file named “07039-2223WO1_SL.xml.” The XML file, created on Apr. 26, 2024, is 2,169 bytes in size. The material in the XML file is hereby incorporated by reference in its entirety.

### TECHNICAL FIELD

This document relates to methods and materials involved in treating cancer. For example, this document provides methods and materials for using antigen presenting cells (APCs; e.g., dendritic cells) (a) designed to release a viral vector that can infect T cells (e.g., an infectious retroviral vector or an infectious lentiviral vector), that is replication-defective within T cells, and that can drive expression of an antigen receptor (e.g., a chimeric antigen receptor (CAR)) and optionally (b) designed to express and/or present one or more foreign antigens (e.g., one or more antigens foreign to the mammal being treated) to treat a mammal (e.g., a human) having cancer.

### BACKGROUND INFORMATION

The rather modest efficacy of T cells expressing a CAR (CAR T cells) against solid tumors derives from multiple immune suppressive mechanisms in the tumor microenvironment, which restrict CAR T cell infiltration, persistence, and function (Newick et al., Annu. Rev. Med., 68:139-152 (2017); Schmidts et al., Front. Immunol., 9:2593 (2018); Morgan et al., Front. Immunol., 9:2493 (2018); and Labanich et al., Nat. Biomed. Eng., 2 (6): 377-391 (2018)).

### SUMMARY

This document provides methods and materials involved in treating cancer. For example, this document provides methods and materials for using APCs (e.g., dendritic cells) designed to release a viral vector that can infect a T cell (e.g., an infectious retroviral vector or an infectious lentiviral vector) and drive expression of an antigen receptor (e.g., a CAR) within that T cell and optionally designed to express and/or present one or more foreign antigens to T cells within a mammal (e.g., a human) having cancer to treat that mammal. In some cases, a mammal (e.g., a human such as a human having cancer) can be administered a population of APCs (e.g., dendritic cells) (a) designed to release a viral vector that can infect a T cell (e.g., an infectious retroviral vector or an infectious lentiviral vector) and drive expression of an antigen receptor (e.g., a CAR) within that T cell and (b) designed to express one or more foreign antigens that can be presented to T cells within the mammal by APCs of the administered population and/or by other APCs within the mammal. In such cases, at least some of the administered APCs can produce and release viral vectors that infect native T cells within the mammal to form CAR⁺ T cells. In addition, the one or more foreign antigens can activate at least some of those infected CAR⁺ T cells via their endogenous T cell receptor (TCR) to form activated dual specific T cells capable of forming dual specific memory T cells that are specific for the foreign antigen via their endogenous TCR and specific for the antigen targeted by the CAR. Such dual specific memory T cells can be stimulated by one or more subsequent administrations of an antigenic composition containing the foreign antigen (e.g., one or more boosters) to generate a potent population of dual specific CAR⁺ effector T cells and/or dual specific CAR⁺ memory T cells within the mammal. Such a potent population of dual specific CAR⁺ effector T cells and/or dual specific CAR⁺ memory T cells can result in effective anti-cancer responses within the mammal, thereby treating cancer.

In some cases, a mammal (e.g., a human such as a human having cancer) can be administered (a) a population of APCs (e.g., dendritic cells) designed to release a viral vector that can infect a T cell (e.g., an infectious retroviral vector or an infectious lentiviral vector) and drive expression of an antigen receptor (e.g., a CAR) within that T cell and (b) a first antigenic composition containing one or more antigens that can be presented to T cells within the mammal by APCs of the administered population and/or by other APCs within the mammal. In such cases, at least some of the administered APCs can produce and release viral vectors that infect native T cells within the mammal to form CAR⁺ T cells. In addition, the presentation of one or more antigens of the first antigenic composition to T cells within the mammal can activate at least some of those infected CAR⁺ T cells via their endogenous TCR to form activated dual specific T cells capable of forming dual specific memory T cells that are specific for the foreign antigen via their endogenous TCR and specific for the antigen targeted by the CAR. Such dual specific memory T cells can be stimulated by one or more subsequent administrations of a second antigenic composition containing the foreign antigen (e.g., one or more boosters) to generate a potent population of dual specific CAR⁺ effector T cells and/or dual specific CAR⁺ memory T cells within the mammal. Such a potent population of dual specific CAR⁺ effector T cells and/or dual specific CAR⁺ memory T cells can result in effective anti-cancer responses within the mammal, thereby treating cancer.

In some cases, a mammal (e.g., a human such as a human having cancer) can be administered a population of allogenic APCs (e.g., allogenic dendritic cells) designed to release a viral vector that can infect a T cell (e.g., an infectious retroviral vector or an infectious lentiviral vector) and drive expression of an antigen receptor (e.g., a CAR) within that T cell. In such cases, at least some of the administered allogenic APCs can produce and release viral vectors that infect native T cells within the mammal to form CAR⁺ T cells. In addition, the allogenic APCs can activate at least some of those infected CAR⁺ T cells via their endogenous TCR to form activated dual specific T cells capable of forming dual specific memory T cells that are specific for a particular antigen via their endogenous TCR and specific for the antigen targeted by the CAR. Such dual specific memory T cells optionally can be stimulated by one or more subsequent administrations of allogenic APCs (e.g., one or more allogenic APC boosters) to generate a potent population of dual specific CAR⁺ effector T cells and/or dual specific CAR⁺ memory T cells within the mammal. Such a potent population of dual specific CAR⁺ effector T cells and/or dual specific CAR⁺ memory T cells can result in effective anti-cancer responses within the mammal, thereby treating cancer. In some cases, a potent population of dual specific CAR⁺ T cells (e.g., dual specific CAR⁺ effector T cells and/or dual specific CAR⁺ memory T cells) can be generated and maintained within the mammal (e.g., human) without a subsequent administration of allogenic APCs after the initial administration of allogenic APCs. For example, a potent anti-cancer response can be triggered within a mammal (e.g., a human) having cancer by administering a population of allogenic APCs (e.g., allogenic dendritic cells) designed to release a viral vector that can infect a T cell (e.g., an infectious retroviral vector or an infectious lentiviral vector) and drive expression of an anti-cancer antigen receptor (e.g., a CAR targeted against the cancer) within that T cell. In such cases, at least some of the administered allogenic APCs can produce and release viral vectors that infect native T cells within the mammal to form CAR⁺ T cells. In addition, the allogenic APCs can activate at least some of those infected CAR⁺ T cells via their endogenous TCR to form activated dual specific T cells capable of forming dual specific memory T cells that are specific for a particular antigen via their endogenous TCR and specific for the cancer antigen targeted by the CAR. In some cases, the immune response that includes these activated dual specific T cells can result in the reduction of the number of cancer cells within the mammal in the absence of any subsequent administration of allogenic APCs.

CAR T cells prepared in vitro can be highly differentiated, short-lived effector cells with a largely exhausted phenotype. CAR TEFF can lack the ability to differentiate in vivo from CAR TN through CAR T_{SCM}, CAR T_{CM}, CAR T_{EM}, and CAR T _{TRM} to allow for generation of a self-perpetuating, persistent, memory effector population. As described herein, administering a population of APCs (e.g., dendritic cells) designed to produce and release a viral vector (e.g., a lentiviral vector) that can infect T cells in vivo, be replication-defective within the infected T cells, and drive expression of a CAR within the infected T cells together with an antigenic stimulation (e.g., administration of an antigenic composition containing, for example, one or more antigens and/or one or more oncolytic viruses) can result in the in vivo generation of naïve CAR⁺ T cells that can recognize both (i) the target of the CAR (e.g., a cancer cell) via the CAR and (ii) an antigen of the antigenic stimulation via an endogenous TCR specific for an antigen of the antigenic composition. Also as demonstrated herein, the naïve CAR⁺ T cells generated within a mammal can differentiate in vivo through the spectrum of T cell memory and effector phenotypes, and dual specific CAR⁺ memory T cells can be reactivated by administering a subsequent antigenic stimulation to direct powerful immune responses (e.g., populations of CAR⁺ effector T cells and/or CAR⁺memory T cells) against the target of the CAR (e.g., cancer). In some cases, dual specific CAR⁺ memory T cells differentiated from in vivo generated naïve CAR⁺ T cells as described herein can be stimulated quickly and effectively to generate populations of CAR⁺ effector T cells and/or CAR⁺ memory T cells that target the targets of the CAR by subsequently administering one or more of the antigens recognized by the endogenous TCR of those dual specific CAR⁺ memory T cells. In such cases, one or more boosts can stimulate the dual specific CAR⁺ memory T cells via their endogenous TCR that is specific for an antigen of the antigenic composition, and they can be free to hunt and kill and/or to generate dual specific CAR⁺ effector T cells that can hunt and kill the CAR targets via their provided CAR.

Also as demonstrated herein, a population of allogenic APCs (e.g., allogenic dendritic cells such as human allogenic dendritic cells) designed to release a viral vector that can infect a T cell (e.g., an infectious retroviral vector or an infectious lentiviral vector) and drive expression of an antigen receptor (e.g., a CAR) within that T cell can be administered to a mammal (e.g., a human) having a disease (e.g., a cancer) that can be treated with activated T cells expressing that antigen receptor (e.g., that CAR) with or without a subsequent administration of any allogenic APCs to treat that disease (e.g., cancer). For example, a mammal (e.g., a human) having cancer can be administered a population of allogenic APCs (e.g., allogenic dendritic cells such as human allogenic dendritic cells) designed to release a viral vector that can infect a T cell (e.g., an infectious retroviral vector or an infectious lentiviral vector) and drive expression of an antigen receptor (e.g., a CAR) within that T cell in a manner that results in the treatment of that cancer in the absence of any subsequent administration of allogenic APCs to that mammal.

The ability to generate CAR⁺ T cells (e.g., naïve CAR⁺ T cells) in a mammal as described herein provides a unique opportunity to use immunotherapy to target (e.g., to locate and destroy) cancer cells, including cancer cells in solid tumors, which can be undetectable by the immune system, and cancer cells at secondary (e.g., metastatic) locations. For example, generating naïve CAR⁺ T cells, activated dual specific CAR⁺ T cells, and dual specific CAR⁺ memory T cells in vivo as described herein can result in T cells that are more active against cancer cells, that persist longer in vivo than conventional CAR⁺ T cells used in current immunotherapies, and that can be rapidly re-activated in vivo to generate CAR⁺ effector T cells via a subsequent administration of a boosting antigen, thereby resulting in long-term anti-cancer responses.

In general, one aspect of this document features methods for treating a mammal having cancer. The methods can include, or consist essentially of, (a) administering a population of antigen presenting cells (APCs) to a mammal having cancer, where the APCs (i) comprise nucleic acid encoding a viral vector including a nucleic acid sequence encoding a chimeric antigen receptor (CAR) targeting a cancer antigen of the cancer and (ii) release a population of the viral vectors within the mammal, where viral vectors of the population of released viral vectors infect a population of T cells within the mammal and are replication-defective within the infected T cells, where the infected T cells express the CAR, (b) administering a first antigenic composition to the mammal, where at least some of the infected T cells expressing the CAR recognize an antigen of the first antigenic composition via an endogenous T cell receptor (TCR) of the infected T cell and form a dual specific memory T cell within the mammal, and (c) administering a second antigenic composition comprising the antigen to the mammal, where the dual specific memory T cell can be stimulated via its endogenous TCR to form dual specific effector T cells comprising the CAR, and where the effector T cells reduce the number of cancer cells within the mammal. The mammal can be a human. The cancer can be a brain stem glioma, a pancreatic cancer, a bile duct cancer, a lung cancer, a skin cancer, a prostate cancer, a breast cancer, an ovarian cancer, a liver cancer, a colorectal cancer, a germ cell tumor, a hepatocellular carcinoma, a bowel cancer, a multiple myeloma, a lymphoma, or a leukemia. The population of APCs can include dendritic cells. The cancer antigen can be cluster of differentiation 19 (CD19), CD22, CD20, GD2, EGFRvIII, mesothelin, IL-13RA, BCMA, CD138, NKG2-D, HER2/Neu, IL-13RA2, CD137, CD28, B7-H3 (CD276), CD16V, CA-125, MUC-1, epithelial tumor antigen, melanoma-associated antigen, mutated p53, mutated Ras, ERBB2, folate binding protein, HIV-1 envelope glycoprotein gp120, HIV-1 envelope glycoprotein gp41, CD123, CD23, CD30, CD56, c-Met, GD3, HERV-K, IL-11R alpha, kappa chain, lambda chain, CSPG4, or VEGFR2. The first antigenic composition can include a virus. The virus can be an oncolytic virus. The virus can be a vesiculovirus, rhabdovirus, reovirus, adenovirus, vaccinia virus, Newcastle disease virus, poliovirus, paramyxoviridae virus, coxsackievirus, senecavirus, herpesvirus, or morbillivirus. The first antigenic composition can include a virus expressing an antigen heterologous to the virus. The first antigenic composition can include an antigenic polypeptide foreign to the mammal. The population of APCs and the first antigenic composition can be administered to the mammal at the same time. The population of APCs can be pre-incubated with the first antigenic composition prior to being administered to the mammal. The population of APCs and the first antigenic composition can be administered to the mammal as a single composition. The population of APCs and the first antigenic composition can be administered to the mammal within from about 1 second to about 48 hours of each other. The dual specific memory T cell can be CD69+ and CD103+. The dual specific memory T cell can be a central memory T cell (T_{CM} cell), an effector memory T cell (T_{EM} cell), a terminally differentiated effector memory T cell (T_{EMRA} cell), or a tissue resident memory T cell (T_{RM} cell). The second antigenic composition can be administered to the mammal at least 5 days after the administering of the population of APCs and the administering of the first antigenic composition. The number of the cancer cells within the mammal can be reduced by at least 25 percent following steps (a)-(c). The method can be effective to improve survival of the mammal as compared to a comparable mammal receiving steps (a) and (b) and not receiving step (c). The survival of the mammal can be improved by at least 25 percent as compared to a comparable mammal receiving steps (a) and (b) and not receiving step (c).

In another aspect, this document features methods for generating memory T cells expressing a CAR within a mammal. The methods can include, or consist essentially of, (a) administering a population of APCs to a mammal, where the APCs (i) comprise nucleic acid encoding a viral vector including a nucleic acid sequence encoding the CAR and (ii) release a population of the viral vectors within the mammal, where viral vectors of the population of released viral vectors infect a population of T cells within the mammal and are replication-defective within the infected T cells, where the infected T cells express the CAR, and (b) administering a first antigenic composition to the mammal, where at least some of the infected T cells expressing the CAR recognize an antigen of the first antigenic composition via an endogenous TCR of the infected T cell and form a dual specific memory T cell within the mammal. The mammal can be a human. The population of APCs can include dendritic cells. The administering step (a), step (b), or both can be intravenous administrations. The population of APCs and the first antigenic composition can be administered to the mammal at the same time. The population of APCs can be pre-incubated with the first antigenic composition prior to being administered to the mammal. The population of APCs and the antigenic composition can be administered to the mammal as a single composition. The population of APCs and the first antigenic composition can be administered to the mammal within from about 1 second to about 48 hours of each other. The CAR can target a cancer antigen. The cancer antigen can be CD19, CD22, CD20, GD2, EGFRVIII, mesothelin, IL-13RA, BCMA, CD138, NKG2-D, HER2/Neu, IL-13RA2, CD137, CD28, B7-H3 (CD276), CD16V, CA-125, MUC-1, epithelial tumor antigen, melanoma-associated antigen, mutated p53, mutated Ras, ERBB2, folate binding protein, HIV-1 envelope glycoprotein gp120, HIV-1 envelope glycoprotein gp41, CD123, CD23, CD30, CD56, c-Met, GD3, HERV-K, IL-11R alpha, kappa chain, lambda chain, CSPG4, or VEGFR2. The antigenic composition can include a virus. The virus can be an oncolytic virus. The virus can be a vesiculovirus, rhabdovirus, reovirus, adenovirus, vaccinia virus, Newcastle disease virus, poliovirus, paramyxoviridae virus, coxsackievirus, senecavirus, herpesvirus, or morbillivirus. The antigenic composition can include a virus expressing an antigen heterologous to the virus. The antigenic composition can include an antigenic polypeptide foreign to the mammal. The dual specific memory T cell can be CD69+ and CD103+. The dual specific memory T cell can be a T_{CM} cell, a T_{EM} cell, a T_{EMRA} cell, or a T_{RM} cell. The mammal can have cancer. The cancer can be a brain stem glioma, a pancreatic cancer, a bile duct cancer, a lung cancer, a skin cancer, a prostate cancer, a breast cancer, an ovarian cancer, a liver cancer, a colorectal cancer, a germ cell tumor, a hepatocellular carcinoma, a bowel cancer, a multiple myeloma, a lymphoma, or a leukemia. The method can include administering a second antigenic composition comprising the antigen to the mammal. The dual specific memory T cell can be stimulated by the antigen of the second antigenic composition via its endogenous TCR to form dual specific effector T cells comprising the CAR. The mammal can have cancer, and the dual specific effector T cells can reduce the number of cancer cells within the mammal. The second antigenic composition can be administered to the mammal at least 5 days after the administering of the population of APCs and the administering of the first antigenic composition. The mammal can have cancer, and the number of cancer cells within mammal can be reduced by at least 25 percent after the administering of the second antigenic composition. The mammal can have cancer, and the method can be effective to improve survival of the mammal as compared to a comparable mammal not receiving the population of APCs. The mammal can have cancer, and the survival of the mammal can be improved by at least 25 percent as compared to a comparable mammal not receiving the population of APCs.

In another aspect, this document features a population of APCs. The population of APCs can comprise nucleic acid encoding a viral vector including a nucleic acid sequence encoding a CAR and can be capable of releasing a population of the viral vectors within a mammal, where viral vectors of the population of released viral vectors are capable of infecting a population of T cells within the mammal and are replication-defective within the infected T cells, and where the infected T cells are capable of expressing the CAR. The APCs can be dendritic cells. The APCs can be human APCs. The CAR can target a cancer antigen. The cancer antigen can be CD19, CD22, CD20, GD2, EGFRvIII, mesothelin, IL-13RA, BCMA, CD138, NKG2-D, HER2/Neu, IL-13RA2, CD137, CD28, B7-H3 (CD276), CD16V, CA-125, MUC-1, epithelial tumor antigen, melanoma-associated antigen, mutated p53, mutated Ras, ERBB2, folate binding protein, HIV-1 envelope glycoprotein gp120, HIV-1 envelope glycoprotein gp41, CD123, CD23, CD30, CD56, c-Met, GD3, HERV-K, IL-11R alpha, kappa chain, lambda chain, CSPG4, or VEGFR2. The APCs can be loaded or coated with an antigenic composition. The antigenic composition can include a virus. The virus can be an oncolytic virus. The virus can be a vesiculovirus, rhabdovirus, reovirus, adenovirus, vaccinia virus, Newcastle disease virus, poliovirus, paramyxoviridae virus, coxsackievirus, senecavirus, herpesvirus, or morbillivirus. The antigenic composition can include a virus expressing an antigen heterologous to the virus. The antigenic composition can include an antigenic polypeptide foreign to the mammal.

In another aspect, this document features methods for treating a human having cancer. The methods can include, or consist essentially of, administering a population of human allogenic APCs to said human, wherein said allogenic APCs (i) comprise nucleic acid encoding a viral vector comprising a nucleic acid sequence encoding a CAR targeting a cancer antigen of said cancer and (ii) release a population of said viral vectors within said human, wherein viral vectors of said population of released viral vectors infect a population of T cells within said human and are replication-defective within said infected T cells, wherein said infected T cells express said CAR, wherein at least some of said infected T cells expressing said CAR form dual specific effector T cells comprising said CAR, and wherein said effector T cells reduce the number of cancer cells within said human in the absence of any subsequence administration of human allogenic APCs to said human. The cancer can be a brain stem glioma, a pancreatic cancer, a bile duct cancer, a lung cancer, a skin cancer, a prostate cancer, a breast cancer, an ovarian cancer, a liver cancer, a colorectal cancer, a germ cell tumor, a hepatocellular carcinoma, a bowel cancer, a multiple myeloma, a lymphoma, or a leukemia. The population of allogenic APCs can include allogenic dendritic cells. The cancer antigen can be CD19, CD22, CD20, GD2, EGFRvIII, mesothelin, IL-13RA, BCMA, CD138, NKG2-D, HER2/Neu, IL-13RA2, CD137, CD28, B7-H3 (CD276), CD16V, CA-125, MUC-1, epithelial tumor antigen, melanoma-associated antigen, mutated p53, mutated Ras, ERBB2, folate binding protein, HIV-1 envelope glycoprotein gp120, HIV-1 envelope glycoprotein gp41, CD123, CD23, CD30, CD56, c-Met, GD3, HERV-K, IL-11R alpha, kappa chain, lambda chain, CSPG4, or VEGFR2. The number of said cancer cells within said human can be reduced by at least 25 percent. The method can be effective to improve survival of said human as compared to a comparable human receiving a population of human autologous APCs instead of said population of said human allogenic APCs. The survival of said human can be improved by at least 25 percent as compared to a comparable human receiving a population of human autologous APCs instead of said population of said human allogenic APCs. In some cases, the method does not comprise administering a subsequent administration of human allogenic APCs to said human.

In another aspect, this document features methods for treating a mammal having cancer. The methods can include, or consist essentially of, administering a population of allogenic APCs to said mammal, wherein said allogenic APCs (i) comprise nucleic acid encoding a viral vector comprising a nucleic acid sequence encoding a CAR targeting a cancer antigen of said cancer and (ii) release a population of said viral vectors within said mammal, wherein viral vectors of said population of released viral vectors infect a population of T cells within said mammal and are replication-defective within said infected T cells, wherein said infected T cells express said CAR, wherein at least some of said infected T cells expressing said CAR form dual specific effector T cells comprising said CAR, and wherein said effector T cells reduce the number of cancer cells within said mammal. The mammal can be a human. The cancer can be a brain stem glioma, a pancreatic cancer, a bile duct cancer, a lung cancer, a skin cancer, a prostate cancer, a breast cancer, an ovarian cancer, a liver cancer, a colorectal cancer, a germ cell tumor, a hepatocellular carcinoma, a bowel cancer, a multiple myeloma, a lymphoma, or a leukemia. The population of allogenic APCs can include allogenic dendritic cells. The cancer antigen can be CD19, CD22, CD20, GD2, EGFRVIII, mesothelin, IL-13RA, BCMA, CD138, NKG2-D, HER2/Neu, IL-13RA2, CD137, CD28, B7-H3 (CD276), CD16V, CA-125, MUC-1, epithelial tumor antigen, melanoma-associated antigen, mutated p53, mutated Ras, ERBB2, folate binding protein, HIV-1 envelope glycoprotein gp120, HIV-1 envelope glycoprotein gp41, CD123, CD23, CD30, CD56, c-Met, GD3, HERV-K, IL-11R alpha, kappa chain, lambda chain, CSPG4, or VEGFR2. The number of cancer cells within said mammal can be reduced in the absence of any subsequent administration of allogenic APCs to said mammal. In some cases, the method does not comprise any subsequent administrations of allogenic APCs to said mammal. In some cases, the method can include administering a subsequent administration of allogenic APCs to said mammal. The subsequent administration of allogenic APCs can include administering only allogenic APCs that lack nucleic acid encoding said viral vector. The subsequent administration of allogenic APCs can include administering allogenic APCs comprising nucleic acid encoding said viral vector. The number of said cancer cells within said mammal can be reduced by at least 25 percent. The method can be effective to improve survival of said mammal as compared to a comparable mammal receiving a population of autologous APCs instead of said population of said allogenic APCs. The survival of said mammal can be improved by at least 25 percent as compared to a comparable mammal receiving a population of autologous APCs instead of said population of said allogenic APCs.

In another aspect, this document features methods for generating memory T cells expressing a CAR within a human. The methods can include, or consist essentially of, administering a population of human allogenic APCs to said human, wherein said allogenic APCs (i) comprise nucleic acid encoding a viral vector comprising a nucleic acid sequence encoding said CAR and (ii) release a population of said viral vectors within said human, wherein viral vectors of said population of released viral vectors infect a population of T cells within said human and are replication-defective within said infected T cells, wherein said infected T cells express said CAR, and wherein at least some of said infected T cells expressing said CAR form a dual specific memory T cell within said human in the absence of a subsequent administration of human allogenic APCs to said human. The population of said allogenic APCs comprises allogenic dendritic cells. The administering step can be an intravenous administration. The CAR can target a cancer antigen. The cancer antigen can be CD19, CD22, CD20, GD2, EGFRvIII, mesothelin, IL-13RA, BCMA, CD138, NKG2-D, HER2/Neu, IL-13RA2, CD137, CD28, B7-H3 (CD276), CD16V, CA-125, MUC-1, epithelial tumor antigen, melanoma-associated antigen, mutated p53, mutated Ras, ERBB2, folate binding protein, HIV-1 envelope glycoprotein gp120, HIV-1 envelope glycoprotein gp41, CD123, CD23, CD30, CD56, c-Met, GD3, HERV-K, IL-11R alpha, kappa chain, lambda chain, CSPG4, or VEGFR2. The dual specific memory T cell can be CD69+ and CD103+. The dual specific memory T cell can be selected from the group consisting of central memory T cells (T_{CM} cells), effector memory T cells (T_{EM} cells), terminally differentiated effector memory T cells (T_{EMRA} cells), and tissue resident memory T cells (T_{RM} cells). The human can have cancer. The cancer can be a brain stem glioma, a pancreatic cancer, a bile duct cancer, a lung cancer, a skin cancer, a prostate cancer, a breast cancer, an ovarian cancer, a liver cancer, a colorectal cancer, a germ cell tumor, a hepatocellular carcinoma, a bowel cancer, a multiple myeloma, a lymphoma, or a leukemia. The number of cancer cells within said human can be reduced by at least 25 percent. The method can be effective to improve survival of said human as compared to a comparable human receiving a population of human autologous APCs instead of said population of said allogenic APCs. The survival of said human can be improved by at least 25 percent as compared to a comparable human receiving a population of human autologous APCs instead of said population of said allogenic APCs. In some cases, the method does not comprise administering a subsequent administration of human allogenic APCs to said human.

In another aspect, this document features methods for generating memory T cells expressing a CAR within a mammal. The methods can include, or consist essentially of, administering a population of APCs to said mammal, wherein said APCs (i) comprise nucleic acid encoding a viral vector comprising a nucleic acid sequence encoding said CAR and (ii) release a population of said viral vectors within said mammal, wherein viral vectors of said population of released viral vectors infect a population of T cells within said mammal and are replication-defective within said infected T cells, wherein said infected T cells express said CAR, and wherein at least some of said infected T cells expressing said CAR form a dual specific memory T cell within said mammal. The mammal can be a human. The population of said allogenic APCs can include allogenic dendritic cells. The administering step can be an intravenous administration. The CAR can target a cancer antigen. The cancer antigen can be CD19, CD22, CD20, GD2, EGFRvIII, mesothelin, IL-13RA, BCMA, CD138, NKG2-D, HER2/Neu, IL-13RA2, CD137, CD28, B7-H3 (CD276), CD16V, CA-125, MUC-1, epithelial tumor antigen, melanoma-associated antigen, mutated p53, mutated Ras, ERBB2, folate binding protein, HIV-1 envelope glycoprotein gp120, HIV-1 envelope glycoprotein gp41, CD123, CD23, CD30, CD56, c-Met, GD3, HERV-K, IL-11R alpha, kappa chain, lambda chain, CSPG4, or VEGFR2. The dual specific memory T cell can be CD69+ and CD103+. The dual specific memory T cell can be selected from the group consisting of central memory T cells (T_{CM} cells), effector memory T cells (T_{EM} cells), terminally differentiated effector memory T cells (T_{EMRA} cells), and tissue resident memory T cells (T_{RM} cells). The mammal can have cancer. The cancer can be a brain stem glioma, a pancreatic cancer, a bile duct cancer, a lung cancer, a skin cancer, a prostate cancer, a breast cancer, an ovarian cancer, a liver cancer, a colorectal cancer, a germ cell tumor, a hepatocellular carcinoma, a bowel cancer, a multiple myeloma, a lymphoma, or a leukemia. The number of cancer cells within said mammal can be reduced by at least 25 percent. The method can be effective to improve survival of said mammal as compared to a comparable mammal receiving a population of autologous APCs instead of said population of said allogenic APCs. The survival of said mammal can be improved by at least 25 percent as compared to a comparable mammal receiving a population of autologous APCs instead of said population of said allogenic APCs. In some cases, the method does not comprise administering a subsequent administration of allogenic APCs to said mammal.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a schematic showing an exemplary method for using APCs (e.g., dendritic cells) to generate CAR T cells from naïve T cells in vivo.

FIGS. 2A-F. In vitro confirmation that engineered dendritic cells generate CAR and antigen dual specific T cells. DCs were transfected with nucleic acid encoding a retroviral vector designed to express a CAR and loaded with immunogenic SIINFEKL (SEQ ID NO:1) peptide. These DCs activated T cells to make SIINFEKL (SEQ ID NO:1)-positive T cells, CAR T cells, and dual specific CAR⁺/SIINFEKL (SEQ ID NO:1)-positive T cells. FIG. 2A is untransduced DC with CD3 T cells. FIG. 2B is DC transfected with the retroviral packaging plasmid but no CAR vector with CD3 T cells. FIG. 2C is DC transfected with the CAR vector but no packaging plasmid, loaded with SIINFEKL (SEQ ID NO:1) peptide, with CD3 T cells. FIG. 2D is CAR T cells prepared from murine splenocytes. FIG. 2E is DC transfected with the retroviral packaging plasmid and with the CAR vector with CD3 T cells. FIG. 2F is DC transfected with the retroviral packaging plasmid and with the CAR vector, loaded with SIINFEKL (SEQ ID NO:1) peptide, with CD3 T cells.

FIG. 3 is a graph plotting the percent survival at the indicated days of mice with established B16-EGFRviii subcutaneous tumors and treated as indicated. C57B1/6 mice bearing 8 day established subcutaneous B16-EGFRVIII tumors were treated with: no treatment (No DC, No Boost No Immune checkpoint blockade); 10⁷ DC engineered to produce CAR retroviral vector and loaded with wild type CSDE1 (non immunogenic) peptide; and boosted at day 15 iv with CSDE1 peptide and control IgG); DC engineered to produce CAR retroviral vector and loaded with wild type CSDE1 (non immunogenic) peptide; and boosted at day 15 iv with SIINFEKL (SEQ ID NO:1) peptide and anti-PD-1 antibody; 10⁷ CAR T cells and boosted at day 15 iv with SIINFEKL (SEQ ID NO:1) peptide and control IgG; CAR T cells and boosted at day 15 iv with SIINFEKL (SEQ ID NO:1) peptide and anti-PD-1 antibody; DC engineered to produce CAR retroviral vector and loaded with SIINFEKL (SEQ ID NO:1) immunogenic peptide; and boosted at day 15 iv with SIINFEKL (SEQ ID NO:1) peptide and control IgG; DC engineered to produce CAR retroviral vector and loaded with SIINFEKL (SEQ ID NO:1) immunogenic peptide; and boosted at day 15 iv with CSDE1 peptide and control IgG; DC engineered to produce CAR retroviral vector and loaded with SIINFEKL (SEQ ID NO:1) immunogenic peptide; and boosted at day 15 iv with SIINFEKL (SEQ ID NO:1) peptide and anti-PD-1 antibody.

FIG. 4 is a graph plotting the percent survival at the indicated days of mice with established B16-EGFRviii subcutaneous tumors and treated as indicated. C57B1/6 mice bearing 8 day established subcutaneous B16-EGFRvIII tumors were treated with: PBS; 10⁷ CAR T cells; 10⁷ DC engineered to produce CAR retroviral vector and loaded with mgp 100 (non-immunogenic) peptide; 10⁷ DC engineered to produce empty retroviral particles with no CAR retroviral vector and loaded with hgp 100 (immunogenic) peptide; 10⁷ DC engineered to produce CAR retroviral vectors and loaded with hgp100 (immunogenic) peptide.

### DETAILED DESCRIPTION

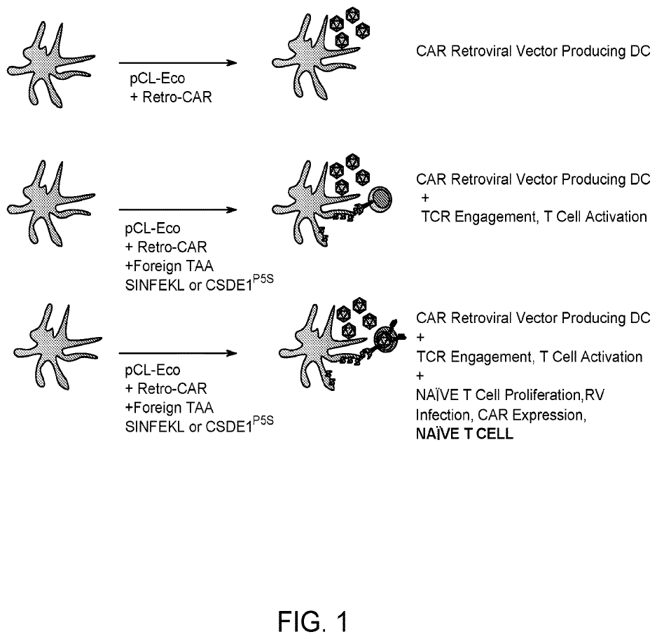

This document provides methods and materials involved in treating cancer. For example, this document provides methods and materials for using a population of APCs (e.g., dendritic cells) designed to produce and release a viral vector (e.g., a lentiviral vector or a retroviral vector) in vivo that contains nucleic acid encoding an antigen receptor (e.g., a CAR), that can infect T cells in vivo, that can be replication-defective within infected T cells, and that can drive expression of the antigen receptor (e.g., a CAR) within infected T cells to generate CAR⁺ T cells within a mammal (e.g., a human). In some cases, the viral vector (e.g., a lentiviral vector or a retroviral vector) can be replication-competent within infected T cells instead of being replication-defective within infected T cells. In some cases, the APCs also can designed to express and/or present one or more antigens (e.g., one or more antigens foreign to the mammal being treated) having the ability to activate at least some of the infected CAR⁺ T cells via their endogenous TCR. In some cases, the APCs can be loaded and/or coated with an antigenic composition containing one or more antigens (e.g., one or more antigens foreign to the mammal being treated) having the ability to activate at least some of the infected CAR⁺ T cells via their endogenous TCR. In some cases, the population of APCs can be administered to the mammal and an antigenic composition containing one or more antigens (e.g., one or more antigens foreign to the mammal being treated) having the ability to activate at least some of the infected CAR⁺ T cells via their endogenous TCR can be administered to the mammal. In each case, the population of APCs (e.g., dendritic cells) designed to produce and release the viral vector can form infected CAR⁺ T cells within the mammal (e.g., naïve CAR⁺ T cells; see, e.g., FIG. 1), and the antigenic stimulation provided by the one or more antigens (e.g., the one or more antigens foreign to the mammal being treated) can activate at least some of the formed infected CAR⁺ T cells via their endogenous TCR to form activated dual specific CAR⁺ T cells. Such activated dual specific CAR⁺ T cells can differentiate within the mammal to form dual specific CAR⁺ memory T cells having the ability to respond quickly to a booster administration containing one or more of the antigens to create dual specific CAR⁺ effector T cells having the ability to kill cancer cells via their CAR.

In some cases, a population of allogenic APCs (e.g., allogenic dendritic cells such as human allogenic dendritic cells) can be designed to release a viral vector that can infect a T cell (e.g., an infectious retroviral vector or an infectious lentiviral vector) and drive expression of an antigen receptor (e.g., a CAR) within that T cell. Such allogenic APCs can be administered to a mammal (e.g., a human) having cancer that is targeted by the antigen receptor (e.g., a CAR) in a manner that can result in the in vivo generation of CAR⁺ T cells (e.g., dual specific CAR⁺ T cells such as dual specific CAR⁺ effector T cells and/or dual specific CAR⁺ memory T cells) as described herein. In some cases, a mammal administered such allogenic APCs can generate a potent anti-cancer response in the absence of any subsequence administration of allogenic APCs. In other cases, subsequent administration of allogenic APCs (e.g., allogenic APCs designed to release a viral vector as described herein; or allogenic APCs that lack the ability to release a viral vector as described herein such unaltered APCs) can result in those dual specific CAR⁺ memory T cells expanding quickly and effectively via stimulation through their endogenous TCRs to generate a population of dual specific CAR⁺ effector T cells that can hunt and kill cells expressing the target of that CAR.

The in vivo generated CAR⁺ T cells (e.g., naïve CAR⁺ T cells, activated dual specific CAR⁺ T cells, dual specific CAR⁺ memory T cells, and/or dual specific CAR⁺ effector T cells) described herein can mediate an immune response against the targets of the CAR. In some cases, the in vivo generated CAR⁺ T cells (e.g., naïve CAR⁺ T cells and/or activated dual specific CAR⁺ T cells) can differentiate into dual specific CAR⁺ memory T cells within the mammal. In such cases, subsequent administration of an antigenic composition can result in those dual specific CAR⁺ memory T cells expanding quickly and effectively via stimulation through their endogenous TCRs to generate a population of dual specific CAR⁺ effector T cells that can hunt and kill cells expressing the target of that CAR. Thus, the methods and material described herein can be used to treat cancer within a mammal.

Any appropriate mammal (e.g., a mammal having cancer) can be treated as described herein. Examples of mammals that can be treated as described herein include, without limitation, humans, non-human primates (e.g., monkeys), dogs, cats, horses, cows, pigs, sheep, mice, rats, and rabbits. In some cases, a human having cancer can be administered a population of APCs (e.g., dendritic cells) as described herein in combination with antigenic stimulation from antigens expressed by the APCs, from antigens loaded and/or coated onto the APCs, and/or from antigens provided by an antigenic composition administered together or separately from the APCs to treat the cancer. In some cases, a human having cancer can be administered a population of allogenic APCs (e.g., allogenic human dendritic cells) as described herein to treat the cancer without any subsequent administrations of allogenic APC for treating the cancer. In some cases, a human having cancer can be administered a population of allogenic APCs (e.g., allogenic human dendritic cells) as described herein to treat the cancer with one or more subsequent administrations of allogenic APC for treating the cancer. In some cases, a mammal (e.g., a human) treated as described herein can be a pediatric mammal (e.g., human less than 18 years of age). In some cases, a mammal (e.g., a human) treated as described herein can be an adult (e.g., a human that is about 60 years of age or older).

Any appropriate population of APCs can be used as described herein. A population of APCs engineered to produce and release a viral vector (e.g., a lentiviral vector) described herein can include any type(s) of APCs. In some cases, a population of APCs described herein can include one, two, three, four, five, or more different types of APCs. For example, a population of APCs can be a heterogeneous population of APCs. In some cases, a population of APCs described herein can be a population of stimulated APCs. In some cases, the APCs of a population of APCs described herein can express MHC class I molecules, MHC class II molecules, or both MHC class I molecules and MHC class II molecules. Examples of APCs that can be used to make a population of APCs described herein include, without limitation, dendritic cells, macrophages, B cells, and Langerhans cells. In some cases, APCs that can be used to make a population of APCs described herein can be obtained from a mammal (e.g., a mammal having cancer). For example, APCs that can be used to make a population of APCs described herein can be obtained from the mammal (e.g., the human) to be treated using the methods and materials described herein. In some cases, APCs that can be used to make a population of APCs described herein can be obtained from a donor mammal (e.g., a donor mammal of the same species) as the mammal to be treated using the methods and materials described herein. For example, when treating a human, APCs that can be used to make a population of APCs described herein can be obtained from a donor human.

In cases where a donor mammal and the mammal to be treated using the methods and materials described herein are humans, the donor human and the human to be treated using the methods and materials described herein can present the same or similar human leukocyte antigens (HLAs; e.g., can be HLA-matched).

A population of APCs described herein can be designed to produce (e.g., can be engineered to produce) and release any appropriate viral vector that contains nucleic acid encoding an antigen receptor (e.g., a CAR), that can infect T cells in vivo, that can be replication-defective within infected T cells, and that can drive expression of the antigen receptor (e.g., a CAR) within infected T cells to generate CAR⁺ T cells within a mammal (e.g., a human). For example, any appropriate viral vector can be designed to contain nucleic acid encoding an antigen receptor (e.g., a CAR), can be designed to infect T cells in vivo, can be designed to be replication-defective within infected T cells, and can be designed to drive expression of the antigen receptor (e.g., a CAR) within infected T cells to generate CAR⁺ T cells within a mammal (e.g., a human) including, without limitation, retroviral vectors, lentiviral vectors, adenoviral vectors, adeno-associated viral vectors, measles virus vectors, replication defective (G-less rhabdovirus vectors), Herpes virus vectors, and vaccinia virus vectors. For example, nucleic acid encoding a viral vector (e.g., a retroviral vector or a lentiviral vector) can be selected and designed to include nucleic acid encoding an antigen receptor (e.g., a CAR) operationally linked to a promotor sequence having the ability to drive expression of the antigen receptor (e.g., a CAR) within T cells. In some cases, such a viral vector can have the ability to infect T cells (e.g., human T cells) without further modification. In cases where a viral vector lacks the natural ability to infect T cells (e.g., human T cells), the viral vector can be modified to add or re-direct tropism to T cells (e.g., human T cells). For example, a viral vector can be modified to include a single-chain variable fragment having the ability to bind to T cells (e.g., an anti-CD3 scFv).

Any appropriate method can be used to create nucleic acid that encodes a viral vector that can be produced in and release from an APC and that is replication-defective in an infected T cell. For example, one or more viral polypeptide needed for viral replication within a T cell can be provided in trans within an APCs designed to express the needed one or more viral polypeptide. In such cases, the APC can release infectious viral vector particles that lack one or more nucleic acid sequences needed for replication within an infected T cell.

Nucleic acid encoding a viral vector (e.g., a retroviral vector or a lentiviral vector) that is included within the APCs of a population of APCs described herein can be designed to include nucleic acid encoding any appropriate antigen receptor (e.g., any appropriate CAR). In some cases, an antigen receptor can be a heterologous antigen receptor. In some cases, an antigen receptor can be a CAR. In some cases, a CAR that targets a cancer or tumor antigen. For example, nucleic acid encoding a viral vector (e.g., a retroviral vector or a lentiviral vector) that is included within the APCs of a population of APCs described herein can be designed to include nucleic acid encoding a CAR that targets a cancer antigen (e.g., a cancer cell surface antigen) expressed by a cancer cell in a mammal having cancer. Examples of antigens that can be the target of such CARs include, without limitation, cluster of differentiation 19 (CD19), CD22, CD20, GD2, EGFRVIII, EGFR, mesothelin, IL-13RA, BCMA, CD138, NKG2-D, HER2/Neu, IL-13RA2, CD137, CD28, B7-H3 (CD276), CD16V, CA-125, MUC-1, epithelial tumor antigen, melanoma-associated antigen, mutated p53, mutated Ras, ERBB2, folate binding protein, HIV-1 envelope glycoprotein gp120, HIV-1 envelope glycoprotein gp41, CD123, CD23, CD30, CD56, c-Met, GD3, HERV-K, IL-11R alpha, kappa chain, lambda chain, CSPG4, and VEGFR2.

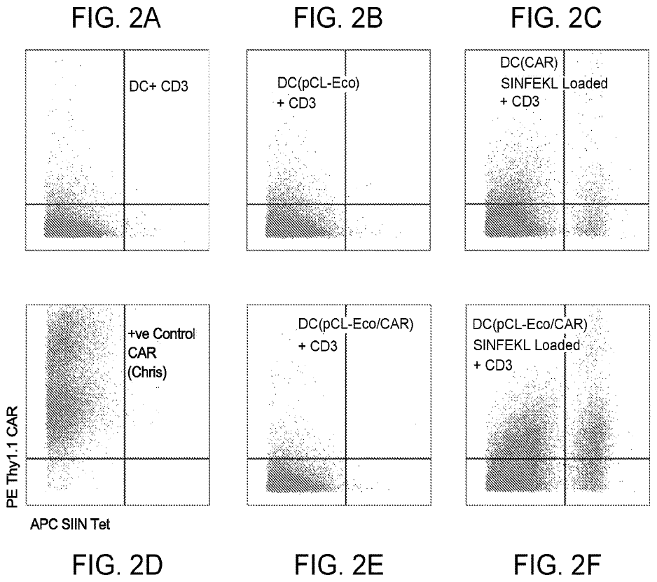

When an antigen receptor is a CAR, the CAR can be any appropriate CAR. A CAR can include an antigen-binding domain, an optional hinge, a transmembrane domain, and one or more signaling domains. An antigen-binding domain of a CAR that can be expressed by a viral vector (e.g., a lentiviral vector) produced and released by an APC as described herein can be any appropriate antigen-binding domain. In some cases, an antigen-binding domain can include an antibody or a fragment thereof that targets an antigen (e.g., a cancer antigen such as a CD19 polypeptide). Examples of antigen-binding domains include, without limitation, an antigen-binding fragment (Fab), a variable region of an antibody heavy (VH) chain, a variable region of a light (VL) chain, a single chain variable fragment (scFv), and domains from growth factors that bind to a cancer cell receptor (e.g., domains from EGF, PDGR, FGF, TGF, or derivatives thereof). In some cases, an antigen-binding domain of a CAR can target (e.g., can target and bind to) a cancer antigen or a cancer-specific antigen. For example, an APC can be designed to produce and release a viral vector (e.g., a lentiviral vector) that can drive expression of a CAR that can bind to a cancer-specific antigen (e.g., an antigen present on cancer cells with minimal, or no, expression on non-cancerous cell types). In some cases, an antigen-binding domain of a CAR can be as described elsewhere (see, e.g., U.S. Patent Application Publication No. 2017/0183418 such as U.S. Patent Application Publication No. 2017/0183418 at paragraph and the sequence listing; U.S. Patent Application Publication No. 2017/0183413 such as U.S. Patent Application Publication No. 2017/0183413 at paragraph [0049], FIG. 2, Table 9, and the sequence listing; U.S. Patent Application Publication No. 2018/0291079 such as U.S. Patent Application Publication No. 2018/0291079 at paragraphs-[0045], and Table 4; U.S. Patent Application Publication No. 2020/0289563 such as U.S. Patent Application Publication No. 2020/0289563 at paragraphs-[0053],-[0189], and Table 1; and U.S. Patent Application Publication No. 2003/0211097 such as U.S. Patent Application Publication No. 2003/0211097 at paragraphs and [0211-0215] and the sequence listing.

In some cases, a CAR described herein can include an optional hinge region. In some cases, a hinge region can be located between an antigen-binding domain and a transmembrane domain of a CAR. In some cases, a hinge region can provide a CAR with increased flexibility for the antigen-binding domain. For example, a hinge region can reduce spatial limitations of an antigen-binding domain of a CAR and its target antigen (e.g., to increase binding between an antigen-binding domain of a CAR and its target antigen). Examples of hinge regions that can be used as described herein include, without limitation, a membrane-proximal region from an IgG, a membrane-proximal region from CD8, and a membrane-proximal region from CD28. In some cases, a hinge region of a CAR can be as described elsewhere (see, e.g., U.S. Patent Application Publication No. 2018/0000914 such as U.S. Patent Application Publication No. 2018/0000914 at paragraph [0168], and Table 1; U.S. Patent Application Publication No. 2017/0183418 such as U.S. Patent Application Publication No. 2017/0183418 at paragraphs [0034], [0037], [0040], and Table 2; U.S. Patent Application Publication No. 2017/0183413 such as U.S. Patent Application Publication No. 2017/0183413 at paragraph [0116]; and U.S. Patent Application Publication No. 2017/0145094 such as U.S. Patent Application Publication No. 2017/0145094 at paragraph [104].

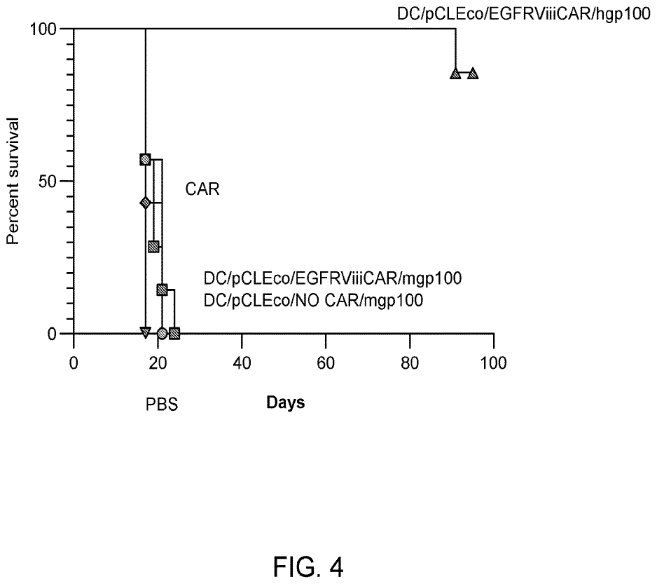

A CAR described herein can include any appropriate transmembrane domain. A transmembrane domain can be located between an antigen-binding domain and a signaling domain of a CAR and/or located between a hinge and a signaling domain of a CAR. In some cases, a transmembrane domain can provide structural stability for the CAR. For example, a transmembrane domain can include a structure (e.g., a hydrophobic alpha helix structure) that can span a cell membrane and can anchor the CAR to the plasma membrane. Examples of transmembrane domains that can be used as described herein include, without limitation, CD32 transmembrane domains, CD4 transmembrane domains, CD8 (e.g., a CD8a) transmembrane domains, CD28 transmembrane domains, CD16 transmembrane domains, and erythropoietin receptor transmembrane domains. In some cases, a transmembrane domain of a CAR can be as described elsewhere (see, e.g., U.S. Patent Application Publication No. 2016/0120906 such as U.S. Patent Application Publication No. 2016/0120906 at paragraphs [0155], [0161], [0269], FIG. 4, and FIG. 11; U.S. Patent Application Publication No. 2019/0209616 such as U.S. Patent Application Publication No. 2019/0209616 at paragraph [0026]; U.S. Patent Application Publication No. 2018/0000914 such as U.S. Patent Application Publication No. 2018/0000914 at paragraphs-; U.S. Patent Application Publication No. 2017/0183418 such as U.S. Patent Application Publication No. 2017/0183418 at paragraphs-[0118]; U.S. Patent Application Publication No. 2017/0183413 such as U.S. Patent Application Publication No. 2017/0183413 at paragraphs-[0118]; and U.S. Patent Application Publication No. 2017/0145094 such as U.S. Patent Application Publication No. 2017/0145094 at paragraphs [104]-[0107].

A CAR described herein can include any appropriate signaling domain or combination of signaling domains (e.g., a combination of two, three, or four signaling domains). In some cases, a signaling domain of a CAR can be an intracellular signaling domain normally found within T cells or NK cells. Examples of signaling domains that can be used as described herein include, without limitation, CD2 signaling domains, CD3ζ C. signaling domains, CD28 signaling domains, Toll-like receptor (TLR) signaling domains (e.g., TLR3 or TLR4 signaling domains), CD27 intracellular signaling domains, OX40 (CD134) intracellular signaling domains, 4-1BB (CD137) intracellular signaling domains, CD278 intracellular signaling domains, DAP10 intracellular signaling domains, DAP12 intracellular signaling domains, FceR1y intracellular signaling domains, CD278 intracellular signaling domains, CD122 intracellular signaling domains, CD132 intracellular signaling domains, CD70 intracellular signaling domains, cytokine receptor intracellular signaling domains, and CD40 intracellular signaling domains. In some cases, a CAR for use as described herein can be designed to be a first generation CAR having a CD35 intracellular signaling domain. In some cases, a CAR for use as described herein can be designed to be a second generation CAR having a CD28 intracellular signaling domain followed by a CD3ζ C. intracellular signaling domain. In some cases, a CAR for use as described herein can be designed to be a third generation CAR having (a) a CD28 intracellular signaling domain followed by (b) a CD27 intracellular signaling domain, an OX40 intracellular signaling domains, or a 4-1BB intracellular signaling domain followed by (c) a CD3} intracellular signaling domain. In some cases, the intracellular signaling domain(s) of a CAR can be as described elsewhere (see, e.g., U.S. Patent Application Publication No. 2018/0000914 such as U.S. Patent Application Publication No. 2018/0000914 at paragraphs-[0167]; and U.S. Patent Application Publication No. 2017/0183413 such as U.S. Patent Application Publication No. 2017/0183413 at paragraphs-[0115].

Examples of CARs that can be expressed by a viral vector (e.g., a lentiviral vector) described herein include, without limitation, EGFRVIII CARs, GD2 CARS, IL-13RA CARS, CD19 CARs, BCMA CARs, CD138 CARs, NKG2-D CARS, HER2 CARS, CD137 CARS, and B7-H3 CARs. Exemplary amino acid sequences for CARs that can be used as described herein are set forth in FIG. 5.

Any appropriate method can be used to engineer an APC (e.g., a dendritic cell) to produce and release a viral vector (e.g., a lentiviral vector) that contains nucleic acid encoding an antigen receptor (e.g., a CAR), that can infect T cells in vivo, that can be replication-defective within infected T cells, and that can drive expression of the antigen receptor (e.g., a CAR) within infected T cells to generate CAR⁺ T cells within a mammal (e.g., a human). For example, nucleic acid encoding such a viral vector can be introduced into an APC by transduction (e.g., viral transduction) or transfection. In some cases, nucleic acid encoding a viral vector described herein can be introduced in vitro or ex vivo into one or more APCs.

Any number of APCs (e.g., dendritic cells) of a population of APCs described herein can be designed to produce and release a viral vector (e.g., a lentiviral vector) that contains nucleic acid encoding an antigen receptor (e.g., a CAR), that can infect T cells in vivo, that can be replication-defective within infected T cells, and that can drive expression of the antigen receptor (e.g., a CAR) within infected T cells to generate CAR⁺ T cells within a mammal (e.g., a human). In some cases, at least half of the APCs within a population of APCs described herein can produce and release such viral vectors. In some cases, from about 25 percent to about 100 percent (e.g., about 50 percent to about 100 percent, about 75 percent to about 100 percent, about 85 percent to about 100 percent, about 95 percent to about 100 percent, or about 75 percent to about 95 percent) of the APCs within a population of APCs described herein can produce and release such viral vectors.

In some cases, the APCs of a population of APCs described herein can be designed to produce and release viral vectors that each contain nucleic acid encoding the same antigen receptor (e.g., a CAR). In some cases, the APCs of a population of APCs described herein can be designed such that some APCs produce and release a viral vector that contains nucleic acid encoding a first antigen receptor (e.g., a first CAR) and some APCs produce and release a viral vector that contains nucleic acid encoding a second antigen receptor (e.g., a second CAR) that is different from the first. In some cases, three, four, five, six, seven, eight, nine, ten, or more different antigen receptors (e.g., CARs) can be provided.

In some cases, an individual APC (e.g., an individual dendritic cell) in a population of APCs described herein can be designed to produce and release two or more (e.g., two, three, four, five, or more) different viral vectors where a first viral vector is designed to encode a first CAR and a second viral vector is designed to encode a second CAR that is different from the first CAR.

As described herein, a population of APCs described herein can be used to form CAR⁺ T cells within a mammal (e.g., a human). As also described herein, antigenic stimulation can be used to activate at least some of the viral vector infected CAR⁺ T cells. Any type of antigenic composition (or antigen) can be used to stimulate viral vector infected CAR⁺ T cells via their endogenous TCR. For example, an antigenic composition described herein can include a virus (e.g., an oncolytic virus). In some cases, an antigenic composition described herein can include a virus that is replication competent. In some cases, an antigenic composition described herein can include a non-pathogenic (e.g., non-pathogenic to a mammal being treated) virus. For example, an antigenic composition described herein can include a virus genetically modified to render it non-pathogenic to a human to be treated. In some cases, an antigenic composition described herein can include a virus that can infect dividing cells (e.g., can infect only dividing cells). In some cases, an antigenic composition described herein can include a virus that can infect non-dividing cells (e.g., can infect only non-dividing cells). In some cases, an antigenic composition described herein can include a virus that contains fusogenic activity. Examples of viruses that can be included in an antigenic composition described herein include, without limitation, rhabdoviruses (e.g., vesiculoviruses (VSVs) and Maraba viruses), reoviruses, adenoviruses, vaccinia viruses, Newcastle disease viruses, polioviruses, herpesviruses (e.g., HSV), and measles viruses.

In some cases, when an antigenic composition described herein includes virus (e.g., an oncolytic virus), the virus can express (e.g., can be designed to express) one or more antigens heterologous to that virus. In some cases, a heterologous antigen expressed by a virus can be a polypeptide that is not endogenous to the mammal being treated. In some cases, a heterologous antigen expressed by a virus can be a full-length polypeptide. In some cases, a heterologous antigen expressed by a virus can be a fragment of a full-length polypeptide (e.g., provided that the fragment retains an antigenic property within a mammal being treated). In some cases, a heterologous antigen expressed by a virus can be derived from a full-length polypeptide (e.g., provided that the fragment retains an antigenic property within a mammal being treated). Examples of that can be a heterologous antigen to a particular virus and that can be used as described herein include, without limitation, ovalbumin polypeptides (OVA) and antigenic fragments thereof, TYRP1 polypeptides and antigenic fragments thereof, TYRP2 polypeptides and antigenic fragments thereof, tyrosinase polypeptides and antigenic fragments thereof, CEA polypeptides and antigenic fragments thereof, MART1 polypeptides and antigenic fragments thereof, MART2 polypeptides and antigenic fragments thereof, SARS-COV-2 spike polypeptides and antigenic fragments thereof, VSV-G polypeptides and antigenic fragments thereof, reovirus surface polypeptides and antigenic fragments thereof, adenovirus coat polypeptides and antigenic fragments thereof, CSDE1 polypeptides and antigenic fragments thereof, and superantigen polypeptides (e.g., Streptococcal pyrogenic exotoxins (SPE), Staphylococcal enterotoxins (SE), and enterotoxogenic E. coli (ETEC) enterotoxins) and antigenic fragments thereof.

A virus expressing one or more antigens (e.g., a heterologous antigen to that virus) can be generated using any appropriate method. In some cases, nucleic acid encoding an antigen (e.g., a heterologous antigen) can be introduced into the genome of a virus such that the antigen is expressed. Nucleic acid encoding an antigen (e.g., a heterologous antigen) can be introduced in the genome of a virus using any appropriate method. In some cases, nucleic acid encoding an antigen (e.g., a heterologous antigen) can be introduced into the genome of a virus by homologous recombination techniques, molecular cloning, and gene editing techniques (e.g., the CRISPR-Cas9 System). Similar methods can be used to introduce nucleic acid encoding an antigen receptor (e.g., a CAR) into the viral vector encoding sequences.

In some cases, an antigenic composition described herein can include an antigenic polypeptide. For example, an antigenic composition described herein can include an antigenic polypeptide that is not endogenous to the mammal being treated. In some cases, an antigenic composition described herein can include a full-length antigenic polypeptide. In some cases, an antigenic composition described herein can include a fragment of a full-length polypeptide (e.g., provided that the fragment retains an antigenic property within the mammal being treated). In some cases, an antigenic composition described herein can include an antigenic polypeptide derived from a full-length polypeptide (e.g., provided that the fragment retains an antigenic property within the mammal being treated). In some cases, an antigenic composition described herein can include an antigenic polypeptide that is foreign (e.g., exogenous) to a mammal (e.g., a human) to be treated. In some cases, an antigenic composition described herein can include an antigenic polypeptide that has no natural counterparts in the mammal (e.g., the human) to be treated. In some cases, an antigenic composition described herein can include a synthetic polypeptide (e.g., a synthetic polypeptide designed to be a potent immunogenic polypeptide). In some cases, an antigenic composition described herein can include an antigenic polypeptide that has no natural counterparts in nature. Examples of antigenic polypeptides that can be included in an antigenic composition described herein include, without limitation, ovalbumin polypeptides (OVA) and antigenic fragments thereof, TYRP1 polypeptides and antigenic fragments thereof, TYRP2 polypeptides and antigenic fragments thereof, tyrosinase polypeptides and antigenic fragments thereof, CEA polypeptides and antigenic fragments thereof, MART1 polypeptides and antigenic fragments thereof, MART2 polypeptides and antigenic fragments thereof, SARS-COV-2 spike polypeptides and antigenic fragments thereof, VSV-G polypeptides and antigenic fragments thereof, reovirus surface polypeptides and antigenic fragments thereof, adenovirus coat polypeptides and antigenic fragments thereof, CSDE1 polypeptides and antigenic fragments thereof, and superantigen polypeptides (e.g., Streptococcal pyrogenic exotoxins (SPE), Staphylococcal enterotoxins (SE), and enterotoxogenic E. coli (ETEC) enterotoxins) and antigenic fragments thereof. In some cases, an antigenic composition described herein can contain one or more antigens of interest in the absence of any virus particles.

In some cases, an antigenic composition described herein (e.g., a composition including one or more viruses such as one or more oncolytic viruses, a composition including one or more viruses designed to express one or more antigens of interest, and/or a composition including one or more antigenic polypeptides of interest) can contain one or more antigens other than polypeptides. Examples of antigens other than polypeptides that can be included in an antigenic composition described herein include, without limitation, polysaccharides (e.g., type 3 S. pneumoniae polysaccharide (Pn3P) and/or polysaccharides of MUC-1) and lipids.

In some cases, a population of APCs described herein and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered to a mammal at the same time (e.g., in a single composition). In some cases when a population of APCs described herein and an antigenic composition described herein are formulated as a single composition, the APCs can be loaded with the antigenic composition. For example, APCs (e.g., dendritic cells) in a population of APCs described herein can be contacted with an antigenic composition containing viruses (e.g., oncolytic viruses) such that the viruses bind to the APCs. In some cases, the viruses (e.g., oncolytic viruses) loaded onto APCs (e.g., dendritic cells) can be covalently bound to the surface of the APCs. In some cases, the viruses (e.g., oncolytic viruses) loaded onto the APCs (e.g., dendritic cells) can be non-covalently bound to the surface of the APCs. In some cases, the viruses (e.g., oncolytic viruses) loaded onto the APCs (e.g., dendritic cells) can be bound to the surface of the APCs through envelope receptor interactions, electrostatic interactions, and/or non-specific interactions between the virus and the APC.

In some cases when a population of APCs described herein and an antigenic composition including one or more viruses (e.g., one or more oncolytic viruses) are formulated as a single composition, at least some of the APCs can be infected with virus. For example, APCs (e.g., dendritic cells) in a population of APCs described herein can be contacted with an antigenic composition including one or more viruses (e.g., one or more oncolytic viruses) such that the viruses infect at least some of the APCs within the population of APCs.

In some cases when a population of APCs described herein and an antigenic composition including one or more viruses (e.g., one or more oncolytic viruses) are formulated as a single composition, the population of APCs and the composition containing the viruses can be combined into that single composition in a manner that results in minimal viral infection of the APCs. For example, a population of APCs described herein and an antigenic composition including one or more viruses (e.g., one or more oncolytic viruses) can be combined and incubated at a temperature of about 2° C. to about 8° C. (e.g., about 2° C. to about 6° C., about 2° C. to about 5° C., about 3° C. to about 8° C., about 4° C. to about 8° C., about 3° C. to about 6° C., about 3° C. to about 5° C., or about 4° C.) for 3 hours or less (e.g., 2.5 hours or less, 2 hours or less, 1.5 hours or less, 1 hour or less, or about 1 hour) prior to being administered to the mammal or prior to being frozen for administration to the mammal at a later time. In such cases, the viruses can infect less than about 10 percent (e.g., less than about 9 percent, less than about 8 percent, less than about 7 percent, less than about 7 percent, or less than about 5 percent) of the APCs of the population. For example, when APCs in a population of APCs described herein are loaded with an antigenic composition including one or more viruses, the viruses can infect less than about 5 percent of the APCs of that population.

In some cases when a population of APCs described herein and an antigenic composition including one or more antigenic polypeptides of interest are combined to form a single composition, that single composition can be designed to lack virus particles. For example, a composition including a population of APCs described herein and an antigenic composition including one or more antigenic polypeptides of interest can be washed or otherwise treated or designed to lack the presence of virus particles.

Any appropriate route of administration can be used to administer a population of APCs described herein, an antigenic composition described herein (e.g., a composition including one or more viruses such as one or more oncolytic viruses, a composition including one or more viruses designed to express one or more antigens of interest, and/or a composition including one or more antigenic polypeptides of interest), and/or a second antigenic composition (e.g., a booster composition) described herein to a mammal. For example, a population of APCs described herein, an antigenic composition described herein, and/or a booster antigenic composition described herein can be administered locally or systemically. In some cases, a population of APCs described herein, an antigenic composition described herein, and/or a booster antigenic composition described herein can be designed for parenteral (e.g., subcutaneous, intramuscular, intravenous, intraperitoneal, and intradermal) administration. Compositions suitable for parenteral administration include aqueous and non-aqueous sterile injection solutions that can contain anti-oxidants, buffers, bacteriostats, and solutes which render the composition isotonic with the blood of the intended recipient; and aqueous and non-aqueous sterile suspensions which may include suspending agents and thickening agents.

In some cases, a population of APCs described herein, an antigenic composition described herein, and/or a booster antigenic composition described herein can be administered systemically by intravenous injection to a mammal (e.g., a human).

In some cases, a population of APCs described herein, an antigenic composition described herein, and/or a booster antigenic composition described herein can be administered a mammal (e.g., a human) separately. For example, a population of APCs described herein and an antigenic composition described herein can be administered to a mammal at the same time (e.g., concurrently) as independent compositions. When a population of APCs described herein and an antigenic composition described herein are administered concurrently, the composition including a population of APCs described herein and the antigenic composition described herein can be administered to a mammal within from about 1 second to about 15 minutes (e.g., about 2 seconds to about 15 minutes, about 5 seconds to about 15 minutes, about 10 seconds to about 15 minutes, about 15 seconds to about 15 minutes, about 1 second to about 10 minutes, about 1 second to about 5 minutes, or about 5 seconds to about 10 minutes) of each other.

In some cases, a population of APCs described herein and an antigenic composition described herein can be administered a mammal (e.g., a human) at different times. When a population of APCs described herein and an antigenic composition described herein are administered to a mammal (e.g., a human) at different times, from about 16 minutes to about 48 hours (e.g., about 16 minutes to about 45 hours, about 16 minutes to about 36 hours, about 16 minutes to about 24 hours, about 16 minutes to about 12 hours, about 16 minutes to about 8 hours, about 16 minutes to about 6 hours, about 16 minutes to about 4 hours, about 30 minutes to about 48 hours, about 1 hour to about 48 hours, about 2 hours to about 48 hours, about 4 hours to about 48 hours, about 6 hours to about 48 hours, or 8 hours minutes to about 48 hours) can elapse between each administration.

When a population of APCs described herein and an antigenic composition described herein are administered as separate compositions (e.g., administered concurrently as separate compositions or administered as separate compositions with from about 16 minutes to about 48 hours between each administration), each composition can be administered to a mammal by any appropriate route. In some cases, a population of APCs described herein and an antigenic composition described herein can be administered by the same route. In some cases, a population of APCs described herein and an antigenic composition described herein can be administered by different routes.

As described herein, a population of APCs described herein can be administered to a mammal (e.g., a human) by any appropriate route. For example, a population of APCs described herein can be administered locally or systemically. In some cases, a composition including a population of APCs described herein can be designed for parenteral (e.g., subcutaneous, intramuscular, intravenous, intraperitoneal, and intradermal) administration. In some cases, a population of APCs described herein can be administered via an intra-tumoral administration. Compositions suitable for parenteral administration include aqueous and non-aqueous sterile injection solutions that can contain anti-oxidants, buffers, bacteriostats, and solutes which render the composition isotonic with the blood of the intended recipient; and aqueous and non-aqueous sterile suspensions which may include suspending agents and thickening agents.

As described herein, an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered to a mammal (e.g., a human) by any appropriate route. For example, an antigenic composition described herein can be administered locally or systemically. In some cases, an antigenic composition described herein can be designed for oral or parenteral (e.g., subcutaneous, intramuscular, intravenous, intraperitoneal, and intradermal) administration. In some cases, an antigenic composition described herein can be administered via an intra-tumoral administration. Compositions suitable for parenteral administration include aqueous and non-aqueous sterile injection solutions that can contain anti-oxidants, buffers, bacteriostats, and solutes which render the composition isotonic with the blood of the intended recipient; and aqueous and non-aqueous sterile suspensions which may include suspending agents and thickening agents. The composition can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

In some cases, a population of APCs described herein can be administered by intravenous injection to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by intravenous injection to the mammal.

In some cases, a population of APCs described herein can be administered by intra-tumoral administration to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by intra-tumoral administration to the mammal.

In some cases, a population of APCs described herein can be administered by intravenous injection to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by intratumoral injection to the mammal.

In some cases, a population of APCs described herein can be administered by intravenous injection to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered orally to the mammal.

When a population of APCs described herein and an antigenic composition described herein (e.g., a composition including one or more viruses such as one or more oncolytic viruses, a composition including one or more viruses designed to express one or more antigens of interest, and/or a composition including one or more antigenic polypeptides of interest) are administered as separate compositions (e.g., administered concurrently as separate compositions or administered as separate compositions with from about 0 seconds to about 15 minutes between each administration), the population of APCs can be administered first, and the antigenic composition administered second, or vice versa.

In some cases, administering (a) a population of APCs described herein and (b) an antigenic composition described herein (e.g., a composition including one or more viruses such as one or more oncolytic viruses, a composition including one or more viruses designed to express one or more antigens of interest, and/or a composition including one or more antigenic polypeptides of interest) to mammal (e.g., a human) can be effective to generate T cells (e.g., naïve T cells) expressing the antigen receptor (e.g., naïve CAR⁺ T cells) in vivo. Examples of types of T cells that can be infected by a viral vector produced and released by an APC described herein include, without limitation, naive T cells (e.g., CD4⁺ naive T cells and/or CD8⁺ naive T cells), cytotoxic T cells (e.g., CD4⁺ CTLs and/or CD8⁺ CTLs), tissue resident memory T cells, regulatory T cells, and central memory T cells. In some cases, an activated dual specific CAR⁺ T cell generated in vivo as described herein using a population of APCs described herein and an antigenic composition described herein can differentiate into a memory T cell (e.g., a CAR⁺ memory T cells) within the mammal. Examples of types of memory T cells that can be generated from CAR⁺ T cells generated within a mammal as described herein include, without limitation, central memory CAR⁺ T cells (CAR⁺ T_{CM} cells), effector memory CAR⁺ T cells (CAR⁺ T_{EM} cells), terminally differentiated effector memory CAR⁺ T cells (CAR⁺ T_{EMRA} cells), and tissue resident memory CAR⁺ T cells (CAR⁺ T_{RM}). In some cases, memory CAR⁺ T cells generated within a mammal (e.g., a human) as described herein (e.g., by administering a population of APCs described herein and an antigenic composition described herein) can be more functional against cancer cells present in the mammal (e.g., as compared to T cells such as CAR⁺ T cells that are generated ex vivo). In some cases, memory CAR⁺ T cells generated within a mammal (e.g., a human) as described herein (e.g., by administering a population of APCs described herein and an antigenic composition described herein) can be more functional against cancer cells present in the mammal (e.g., as compared to CAR⁺ T cells that are generated ex vivo) as assessed by, for example, increased cytotoxicity against CAR target cancer cells and/or increased IFN-γ secretion upon stimulation with cancer cells.

In some cases, memory CAR⁺ T cells generated within a mammal (e.g., a human) as described herein (e.g., by administering a population of APCs described herein and an antigenic composition described herein) can persist longer within the mammal (e.g., as compared to CAR″ T cells that are generated ex vivo). For example, the methods and materials described herein can be used to generate CAR⁺ T cells in vivo that can persist within a mammal (e.g., a human) for from about 40 days to about 2 years (e.g., from about 40 days to about 1.5 years, from about 40 days to about 1 year, from about 40 days to about 11 months, from about 40 days to about 10 months, from about 40 days to about 9 months, from about 40 days to about 8 months, from about 40 days to about 7 months, from about 40 days to about 6 months, from about 50 days to about 200 days, from about 50 days to about 180 days, from about 50 days to about 160 days, from about 50 days to about 150 days, from about 50 days to about 125 days, or from about 80 days to about 1 year).

Once memory CAR⁺ T cells are generated within a mammal, the mammal can be administered a second antigenic composition (e.g., a composition including one or more viruses such as one or more oncolytic viruses, a composition including one or more viruses designed to express one or more antigens of interest, and/or a composition including one or more antigenic polypeptides of interest) one or more (e.g., one, two, three, four, five, or more) times. For example, a mammal can be subsequently administered (e.g., can be boosted with) a second antigenic composition from about 5 days to about 5 years (e.g., from about 5 days to about 5 years, from about 7 days to about 5 years, from about 10 days to about 5 years, from about 14 days to about 5 years, from about 21 days to about 5 years, from about 1 month to about 5 years, from about 2 months to about 5 years, from about 3 months to about 5 years, from about 4 months to about 5 years, from about 5 months to about 5 years, from about 6 months to about 5 years, from about 5 days to about 4.5 years, from about 5 days to about 4 years, from about 5 days to about 3.5 years, from about 5 days to about 3 years, from about 5 days to about 2.5 years, from about 5 days to about 2 years, from about 5 days to about 1.5 years, from about 5 days to about 1 year, from about 5 days to about 10 months, from about 5 days to about 8 months, from about 5 days to about 6 months, from about 5 days to about 4 months, from about 5 days to about 3 months, from about 5 days to about 2 months, from about 5 days to about 1 month, from about 5 days to about 20 days, from about 5 days to about 15 days, from about 5 days to about 10 days, from about 10 days to about 200 days, from about 20 days to about 200 days, from about 30 days to about 200 days, from about 40 days to about 200 days, from about 50 days to about 200 days, from about 10 days to about 175 days, from about 10 days to about 150 days, from about 10 days to about 125 days, from about 10 days to about 100 days, from about 50 days to about 110 days, or from about 60 days to about 100 days) after having been administered (a) a population of APCs described herein and (b) a first antigenic composition described herein (e.g., a composition including one or more viruses such as one or more oncolytic viruses, a composition including one or more viruses designed to express one or more antigens of interest, and/or a composition including one or more antigenic polypeptides of interest). In some cases, a mammal can be administered a second antigenic composition (e.g., a boost) from about 5 days to about 150 days (e.g., from about 60 days to about 100 days) after having been administered (a) a population of APCs described herein and (b) a first antigenic composition described herein. For example, a mammal can be administered a second antigenic composition from about 5 days to about 8 days (e.g., about 7 days) after having been administered a population of APCs described herein and a first antigenic composition described herein.

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) can include the same antigen(s) as a first antigenic composition that was administered together with a population of APCs described herein.

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) can include one or more different antigens as compared to the first antigenic composition that was administered together with a population of APCs described herein.

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) can lack APCs (e.g., can lack a population of APCs). For example, a mammal (e.g., a human) can be administered (a) a population of APCs described herein and (b) a first antigenic composition described herein (e.g., a composition including one or more viruses such as one or more oncolytic viruses, a composition including one or more viruses designed to express one or more antigens of interest, and/or a composition including one or more antigenic polypeptides of interest). Then, at least about 5 days (e.g., after at least about 7 days, after at least about 10 days, after at least about 14 days, after at least about 20 days, after at least about 50 days, after at least about 60 days, after at least about 75 days, after at least about 3 months, after at least about 4 months, after at least about 5 months, or after at least about 6 months) after the latter administration of that population of APCs and that first antigenic composition, the mammal (e.g., the human) can be administered a second antigenic composition that does not include APCs. In some cases, that second antigenic composition can be identical to the first antigenic composition administered to the mammal. For example, in some cases, the first antigenic composition administered to the mammal can include one or more oncolytic viruses (e.g., one or more VSV viruses, one or more reoviruses, one or more measles viruses, or combinations thereof), and the second antigenic composition administered to the mammal can include those same one or more oncolytic viruses. In some cases, the second antigenic composition can be different from the first antigenic composition administered to the mammal. For example, in some cases, the first antigenic composition administered to the mammal can include one or more viruses designed to express one or more antigens of interest, and the second antigenic composition administered to the mammal can include one or more of those antigens of interest that were expressed by the viruses of the first antigenic composition with that second antigenic composition lacking the viruses. In some cases, a mammal (e.g., a human) can be treated as described herein with the initially administered population of APCs being the only APCs that are administered to the mammal.

In some cases, a mammal (e.g., a human) can be treated as described herein with the initially administered population of APCs and a first antigenic composition, and can be subsequently treated with multiple rounds of additional populations of APCs described herein and/or additional antigenic compositions.

In some cases, a second antigenic composition can include one or more viruses (e.g., one or more oncolytic viruses). In some cases, a second antigenic can include one or more antigenic polypeptides of interest. For example, a second antigenic composition can include one or more antigenic polypeptides of interest that were expressed by a virus present in a first antigen composition. For example, a second antigenic composition can include one or more antigenic polypeptides of interest that were expressed by a virus present in a first antigen composition and can lack virus particles.

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) can be administered to a mammal (e.g., a human) as the sole active agent to stimulate the memory CAR⁺ T cells generated within the mammal as described herein.

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) can be administered to a mammal (e.g., a human) together with one or more additional agents that can stimulate memory CAR⁺ T cells within the mammal (e.g., can stimulate the memory CAR⁺ T cells generated within the mammal as described herein). Examples of additional agents (e.g., other than a second antigen composition) that can be used to stimulate memory CAR⁺ T cells within a mammal include, without limitation, pathogens and TLR agonists.

A second antigenic composition (e.g., a composition including one or more viruses such as one or more oncolytic viruses, a composition including one or more viruses designed to express one or more antigens of interest, and/or a composition including one or more antigenic polypeptides of interest) can be administered to a mammal by any appropriate route. For example, a second antigenic composition described herein can be administered locally or systemically. In some cases, a second antigenic composition described herein can be designed for oral or parenteral (e.g., subcutaneous, intramuscular, intravenous, intraperitoneal, and intradermal) administration. When being administered orally, a composition can be in the form of a pill, tablet, or capsule. Compositions suitable for parenteral administration include aqueous and non-aqueous sterile injection solutions that can contain anti-oxidants, buffers, bacteriostats, and solutes which render the composition isotonic with the blood of the intended recipient; and aqueous and non-aqueous sterile suspensions which may include suspending agents and thickening agents. The composition can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

In some cases, a second antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by intravenous injection to the mammal.

In some cases, administration of a second antigenic composition (e.g., a composition including one or more viruses such as one or more oncolytic viruses, a composition including one or more viruses designed to express one or more antigens of interest, and/or a composition including one or more antigenic polypeptides of interest) as described herein (e.g., a boost) can be effective to activate memory CAR⁺ T cells generated as described herein. For example, a subsequent administration (e.g., a boost) of an antigenic composition can be used to reactivate rapidly memory CAR⁺ T cells generated by administering a population of APCs described herein and a first antigenic composition described herein to generate naïve CAR⁺ T cells that can differentiate into memory CAR⁺ T cells (e.g., memory CAR⁺ T cells that can recognize an antigen that was present in both the first antigenic composition and the boost).

In some cases, the methods and materials provided herein can be used to treat a mammal (e.g., a human) having cancer. For example, a mammal in need of cancer treatment (e.g., a mammal having cancer) can be administered (a) a population of APCs described herein and (b) an antigenic composition described herein (e.g., a composition including one or more viruses such as one or more oncolytic viruses, a composition including one or more viruses designed to express one or more antigens of interest, and/or a composition including one or more antigenic polypeptides of interest), and after at least about 5 days (e.g., after at least about 7 days, after at least about 10 days, after at least about 14 days, after at least about 20 days, after at least about 50 days, after at least about 60 days, after at least about 75 days, after at least about 3 months, after at least about 4 months, after at least about 5 months, or after at least about 6 months), can be subsequently administered a second antigenic composition (e.g., a composition including one or more viruses such as one or more oncolytic viruses, a composition including one or more viruses designed to express one or more antigens of interest, and/or a composition including one or more antigenic polypeptides of interest) to reduce or eliminate the number of cancer cells present within the mammal. For example, the methods and materials described herein can be used to reduce the number of cancer cells present within a mammal having cancer by, for example, 10, 20, 30, 40, 50, 60, 70, 80, 90, 95, or more percent. For example, the methods and materials described herein can be used to reduce the size (e.g., volume) of one or more tumors present within a mammal having cancer by, for example, 10, 20, 30, 40, 50, 60, 70, 80, 90, 95, or more percent. In some cases, the number of cancer cells present within a mammal being treated can be monitored. Any appropriate method can be used to determine whether or not the number of cancer cells present within a mammal is reduced. For example, imaging techniques can be used to assess the number of cancer cells present within a mammal.

In some cases, the methods and materials provided herein can be used to improve survival of a mammal (e.g., a human) having cancer. For example, a mammal in need cancer treatment (e.g., a mammal having cancer) can be administered (a) a population of APCs described herein and (b) an antigenic composition described herein (e.g., a composition including one or more viruses such as one or more oncolytic viruses, a composition including one or more viruses designed to express one or more antigens of interest, and/or a composition including one or more antigenic polypeptides of interest), and after at least about 5 days (e.g., after at least about 7 days, after at least about 10 days, after at least about 14 days, after at least about 20 days, after at least about 50 days, after at least about 60 days, after at least about 75 days, after at least about 3 months, after at least about 4 months, after at least about 5 months, or after at least about 6 months), can be subsequently administered a second antigenic composition (e.g., a composition including one or more viruses such as one or more oncolytic viruses, a composition including one or more viruses designed to express one or more antigens of interest, and/or a composition including one or more antigenic polypeptides of interest) to improve survival of the mammal. For example, the methods and materials described herein can be used to improve the survival of a mammal having cancer by, for example, 10, 20, 30, 40, 50, 60, 70, 80, 90, 95, or more percent. For example, the methods and materials described herein can be used to improve the survival of a mammal having cancer by, for example, at least 6 months (e.g., about 6 months, about 8 months, about 10 months, about 1 year, about 1.5 years, about 2 years, about 2.5 years, about 3 years, about 4 years, about 5 years, or more).

In some cases, the methods described herein also can include identifying a mammal as having cancer. Examples of methods for identifying a mammal as having cancer include, without limitation, physical examination, laboratory tests (e.g., blood and/or urine), biopsy, imaging tests (e.g., X-ray, PET/CT, MRI, and/or ultrasound), nuclear medicine scans (e.g., bone scans), endoscopy, and/or genetic tests. Once identified as having cancer, a mammal can be administered or instructed to self-administer (a) a population of APCs described herein and (b) an antigenic composition described herein (e.g., a composition including one or more viruses such as one or more oncolytic viruses, a composition including one or more viruses designed to express one or more antigens of interest, and/or a composition including one or more antigenic polypeptides of interest). Then, in some cases, at least about 5 days (e.g., after at least about 7 days, after at least about 10 days, after at least about 14 days, after at least about 20 days, after at least about 50 days, after at least about 60 days, after at least about 75 days, after at least about 3 months, after at least about 4 months, after at least about 5 months, or after at least about 6 months) after the latter administration of the population of APCs and the antigenic composition, the mammal can be administered or instructed to self-administer a second antigenic composition that includes at least some of the antigens present in the first antigenic composition administered to the mammal.

The methods and materials described herein can be used to treat a mammal (e.g., a human) having any type of cancer. In some cases, a cancer treated as described herein can include one or more solid tumors. In some cases, a cancer treated as described herein can be a blood cancer. In some cases, a cancer treated as described herein can be a primary cancer. In some cases, a cancer treated as described herein can be a metastatic cancer. In some cases, a cancer treated as described herein can be a refractory cancer. In some cases, a cancer treated as described herein can express a cancer-specific antigen. Examples of cancers that can be treated as described herein include, without limitation, brain cancers (e.g., brain stem gliomas such as high-grade gliomas (HGGs)), pancreatic cancers (e.g., pancreatic adenocarcinoma), bile duct cancers (e.g., cholangiocarcinoma), lung cancers (e.g., mesothelioma), skin cancers (e.g., melanoma), prostate cancers, breast cancers, ovarian cancers, liver cancers, colorectal cancers, germ cell tumors, hepatocellular carcinoma, bowel cancers, multiple myeloma, lymphomas (e.g., B cell lymphomas such as diffuse large cell lymphoma), leukemias (e.g., chronic lymphocytic leukemia (CLL), acute lymphoblastic leukemia (ALL), and acute myeloid leukemia (AML)), and uveal melanoma. In some cases, a cancer treated as described herein can be a brain stem glioma (e.g., a HGG). For example, a cancer treated as described herein can be a brain stem glioma (e.g., a HGG) in a pediatric human.

In some cases, the methods and materials described herein can be used as a combination therapy with one or more additional agents used to treat a cancer. For example, a mammal in need of cancer treatment (e.g., a mammal having cancer) can be administered (a) a population of APCs described herein, (b) a first antigenic composition described herein, and (c) a second antigenic composition as a subsequent boost as described herein, in combination with one or more anti-cancer treatments. Examples of anti-cancer treatments that can be used in combination with the administrations of APC populations described herein and antigenic compositions described herein include, without limitation, cancer surgeries, radiation therapies, chemotherapies (e.g., chemotherapies with alkylating agents such as busulfan), checkpoint blockade therapies (e.g., anti-PD-1 antibody therapy, anti-PD-L1 antibody therapy, and/or anti-CTLA4 antibody therapy), targeted therapies (e.g., GM-CSF inhibiting agents such as lenzilumab), hormonal therapies, angiogenesis inhibitors, immunosuppressants (e.g., interleukin-6 inhibiting agents such as tocilizumab), and cytokine release syndrome (CRS) treatments (e.g., ruxolitinib or ibrutinib). In cases where the methods and materials described herein are used in combination with additional agents treat a cancer, the one or more additional agents can be administered at the same time or independently. In some cases, the methods and materials described herein can be administered or performed first, and the one or more additional agents administered second, or vice versa.

In some cases, the methods and materials described herein can be used to treat a mammal having a disease, disorder, or condition other than cancer. For example, a mammal having a disease, disorder, or condition other than cancer can be administered (a) a population of APCs described herein and (b) a first antigenic composition (e.g., a composition including one or more viruses such as one or more oncolytic viruses, a composition including one or more viruses designed to express one or more antigens of interest, and/or a composition including one or more antigenic polypeptides of interest), and after at least about 5 days (e.g., after at least about 7 days, after at least about 10 days, after at least about 14 days, after at least about 20 days, after at least about 50 days, after at least about 60 days, after at least about 75 days, after at least about 3 months, after at least about 4 months, after at least about 5 months, or after at least about 6 months), can be subsequently administered a second antigenic composition (e.g., a composition including one or more viruses such as one or more oncolytic viruses, a composition including one or more viruses designed to express one or more antigens of interest, and/or a composition including one or more antigenic polypeptides of interest). In such cases, the APCs can be designed to produce and release a viral vector (e.g., a lentiviral vector) that contains nucleic acid encoding an antigen receptor (e.g., a CAR) that targets an antigen associated with a disease, disorder, or condition instead of an antigen receptor (e.g., a CAR) that targets a cancer antigen. An example of antigens that can be targeted by the methods and materials described herein to target a disease, disorder, or condition other than cancer include, without limitation, an urokinase-type plasminogen activator receptor (uPAR) antigen to treat conditions associated with senescence.

### Examples

### Example 1: In Vitro Assessment of Engineered Dendritic Cells for Producing Naive CAR T Cells In Vivo for Cancer Therapy

### Methods

Murine CD14 activated DCs were prepared from C57B1/6 mice as follows. Day 1: DCs were transfected with lipofectamine with 5 μg of pCL Eco (Naviaux et al., J. Virol., 70 (8): 5701-5 (1996)), EGFRVIII CAR retroviral vector (Sampson et al., Clin. Cancer Res., 20:972-984 (2014)), or both. DCs only receiving only receiving the EGFRVIII CAR retroviral vector will not release retroviruses and DCs receiving pCL Eco will release empty retroviral particles (no genomes), while DCs receiving both will release retroviruses capable of infecting T cells. Since T cells lack pCL Eco, they will not release retroviruses after being infected with the retroviruses released from the DCs. The infected T cells, however, will express the CAR encoded by the retroviral vector upon proliferation of the naïve T cell (e.g., as it becomes activated through its TCR by the immunogenic peptide presented by the DC). Day 3:10⁶ DCs were loaded with 1 mg of peptide of either SIINFEKL (SEQ ID NO:1) or CSDE1, and were immediately co-cultured with 10⁷ naïve CD3+ T cells that were sorted from splenocytes using magnetic beads. The loading was performed as follows. Briefly, peptide was added to the well at 1 mg/mL. Day 7: Flow cytometry was performed by gating on CD8⁺ cells and analysed for Thy1.1 marker (expressed by the CAR vector) and SIINFEKL (SEQ ID NO:1) tetramer positive cells.

In particular, the following six experimental conditions were used. FIG. 2A is untransduced DC with CD3 T cells. FIG. 2B is DC transfected with the retroviral packaging plasmid but no CAR vector with CD3 T cells. FIG. 2C is DC transfected with the CAR vector but no packaging plasmid, loaded with SIINFEKL peptide, with CD3 T cells. FIG. 2D is CAR T cells prepared from murine splenocytes. FIG. 2E is DC transfected with the retroviral packaging plasmid and with the CAR vector with CD3 T cells. FIG. 2F is DC transfected with the retroviral packaging plasmid and with the CAR vector, loaded with SIINFEKL (SEQ ID NO:1) peptide, with CD3 T cells.

### Results

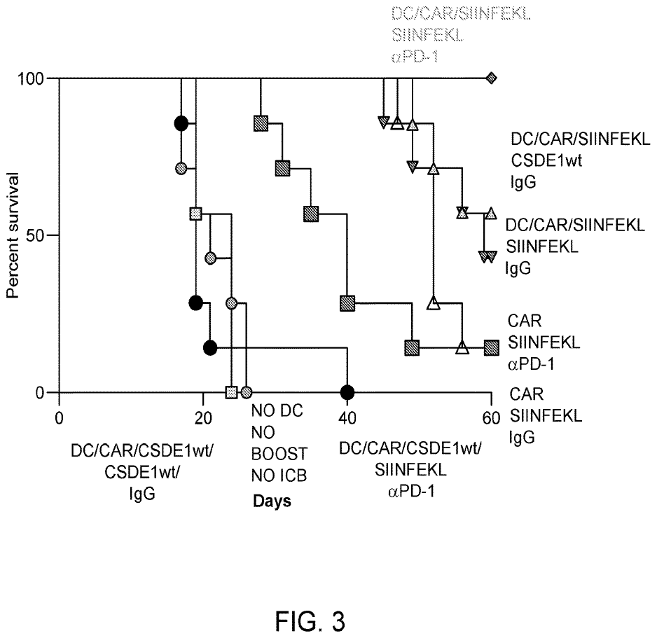

DC loaded with immunogenic SIINFEKL (SEQ ID NO:1) peptide and producing retroviruses designed to express a CAR generated single positive CAR T cells, single positive SIINFEKL (SEQ ID NO:1) T cells, and dual specific CAR/SIINFEKL (SEQ ID NO: 1) T cells (FIG. 3F).

### Example 2: Using Engineered Dendritic Cells to Generate Dual Specific T Cells In Vivo and Treat Cancer

### Methods

### Mouse Xenograft Study of SIINFEKL (SEQ ID NO:1)

C57B1/6 mice bearing 8 day established subcutaneous B16-EGFRvIII tumors were treated with: no treatment (No DC, No Boost No Immune checkpoint blockade); 10⁷ DC engineered to produce CAR retroviral vector and loaded with wild type CSDE1 (non immunogenic) peptide; and boosted at day 15 iv with CSDE1 peptide and control IgG); DC engineered to produce CAR retroviral vector and loaded with wild type CSDE1 (non immunogenic) peptide; and boosted at day 15 iv with SIINFEKL (SEQ ID NO:1) peptide and anti-PD-1 antibody; 10⁷ CAR T cells and boosted at day 15 iv with SIINFEKL (SEQ ID NO: 1) peptide and control IgG; CAR T cells and boosted at day 15 iv with SIINFEKL (SEQ ID NO: 1) peptide and anti-PD-1 antibody; DC engineered to produce CAR retroviral vector and loaded with SIINFEKL (SEQ ID NO:1) (immunogenic) peptide; and boosted at day 15 iv with SIINFEKL (SEQ ID NO:1) peptide and control IgG; DC engineered to produce CAR retroviral vector and loaded with SIINFEKL (SEQ ID NO:1) (immunogenic) peptide; and boosted at day 15 iv with CSDE1 peptide and control IgG; DC engineered to produce CAR retroviral vector and loaded with SIINFEKL (SEQ ID NO:1) (immunogenic) peptide; and boosted at day 15 iv with SIINFEKL (SEQ ID NO:1) peptide and anti-PD-1 antibody.

### Mouse Xenograft Study of Hpg100/Mpg100

C57B1/6 mice bearing 8 day established subcutaneous B16-EGFRvIII tumors were treated with: PBS; 10⁷ CAR T cells; 10⁷ DC engineered to produce CAR retroviral vector and loaded with mgp100 (non-immunogenic) peptide; 10⁷ DC engineered to produce empty retroviral particles with no CAR retroviral vector and loaded with hgp 100 (immunogenic) peptide; 10⁷ DC engineered to produce CAR retroviral vectors and loaded with hgp 100 (immunogenic) peptide.

### Results

When mice with 8 day established subcutaneous tumors were treated with adoptively transferred dendriticc cells which were both producing CAR retroviral vector and were presenting an immunogenic peptide, tumors regressed and, in the majority of cases, were cleared completely (FIGS. 3 and 4). In one case, the immunogen presented by the CAR producing DC was the SIINFEKL (SEQ ID NO:1) peptide from the chicken ovalbumin protein and had no relevance to the tumor per se (FIG. 3). In the second example, the melanomas were treated by presenting the hgp 100 peptide by the CAR vector-producing DC; in this latter case, the hgp100 peptide acts as a heteroclitic antigen in that it activates murine T cells (is immunogenic in the mouse) which also cross react against the corresponding murine epitope which differs in two amino acids from the human epitope. In both FIGS. 3 and 4, control treatments of DC producing CAR retroviral vectors but presenting non-immunogenic (self) peptides to which the mice are tolerant (either wild type CSDE1 (FIG. 3) or mgp100 (FIG. 4)) were ineffective at controlling tumor growth. These data show that only upon immunogenic presentation of peptides to naïve T cells will the CAR vectors released by the DC be able to infect naïve T cells which strat to proliferate as they become activated through their TCR by the immunogens presented by the DC.

### Example 3: Treating Cancer with Engineered Dendritic Cells and an Antigenic Composition

Dendritic cells designed to release a viral vector (e.g., a lentiviral vector) that can infect T cells, that is replication-defective in infected T cells, and that can express a CAR within infected T cells are administered (e.g., systemically administered) to a mammal (e.g., a human) together with an antigenic composition (e.g., an oncolytic virus preparation or composition containing one or more antigens). In some cases, the dendritic cells designed to release the viral vector and the antigenic composition (e.g., oncolytic viruses) are in separate compositions that are co-administered. In some cases, the dendritic cells designed to release the viral vector are loaded (e.g., coated) with the antigenic composition (e.g., oncolytic viruses) and administered together as a single composition.

A boost (e.g., subsequent administration) of the antigenic composition (e.g., an oncolytic virus preparation or composition containing one or more antigens) is administered (e.g., systemically administered) to the mammal about 1 to 3 weeks (e.g., about 1 week) after the initial administration of dendritic cells, thereby producing an effective anti-cancer response within the mammal.

This procedure can result in the generation of dual specific T cell that are specific for the target antigen of the CAR and specific for an epitope of the antigenic composition (see, e.g., FIG. 1). Such dual specific T cells can differentiate into dual specific CAR⁺ T memory cells and/or dual specific CAR⁺ effector T cells within the mammal.

The boost (e.g., systemic boost) with the antigenic composition (or a portion thereof) can re-activate the dual specific CAR⁺ memory T cells in vivo. Those re-activated dual specific CAR⁺ memory T cells can target (e.g., target and destroy) cells (e.g., cancer cells) presenting antigens recognized by the CAR present on the dual specific CAR⁺ memory T cells and/or can generate dual specific effector T cells that are CAR⁺ and that can target (e.g., target and destroy) cells (e.g., cancer cells) presenting antigens recognized by the CAR present on those dual specific CAR⁺ effector T cells.

### Example 4: Treating Cancer with Engineered Dendritic Cells and an Antigenic Composition

The following groups of mice were treated as indicated in Table 1.

TABLE 1

| | | Day 100 |
|---|---|---|
| | | number of |
| Group | Treatment | survivors |
| A | Autologous DCs/CAR/No SIINFEKL/No boost | 0/8 (day 14) |
| B | Viral vector encoding CAR alone | 4/8 |
| C | Allogenic DC/No CAR/No boost | 0/8 (day 21) |
| D | Allogenic DC/CAR/Allogenic DC boost | 4/8 |
| E | Allogenic DC/CAR/SIINFEKL/SIINFEKL Boost | 6/8 |
| F | Autologous DC/CAR/SIINFEKL/No Boost | 4/8 |
| G | Allogenic DC/CAR/No Boost | 7/8 |
| H | Autologous DC/CAR/SIINFEKL/Autologous | 3/8 |
| | DC Boost | |
| I | Autologous DC/CAR/SIINFEKL/SIINFEKL | 6/8 |
| | Boost | |
| J | Allogenic DC/CAR/Allogenic DC Boost | 7/8 |

For those groups treated with DCs, the allogenic or autologous DCs were engineered to encode a replication-defective lentivirus that encodes a CAR or a CAR plus SIINFEKL (SEQ ID NO:1) antigen as indicated. For those groups boosted with allogenic or autologous DCs, the boost included DCs that were not engineered to encode the replication-defective lentivirus.

These results demonstrate that allogenic and autologous DCs engineered to encode a replication-defective lentivirus that encodes a CAR can be used to treat cancer. These results also demonstrate that allogenic and autologous DCs engineered to encode a replication-defective lentivirus that encodes a CAR can be used with one or more antigenic boosts to treat cancer.

### Example 5: Amino acid sequences for exemplary CARs

Exemplary EGFRvIII CAR (SEQ ID NO: 2):
MVLLVTSLLLCELPHPAFLLIPDIQMTQSPSSLSASVGDRVTITCRASQGIRNNLAWYQ
QKPGKAPKRLIYAASNLQSGVPSRFTGSGSGTEFTLIVSSLQPEDFATYYCLQHHSYPL
TSGGGTKVEIKRTGSTSGSGKPGSGEGSEVQVLESGGGLVQPGGSLRLSCAASGFTFS
SYAMSWVRQAPGKGLEWVSAISGSGGSTNYADSVKGRFTISRDNSKNTLYLQMNSL
RAEDTAVYYCAGSSGWSEYWGQGTLVTVSSAAATTTKPVLRTPSPVHPTGTSQPQRP
EDCRPRGSVKGTGLDFACDIYIWAPLAGICVALLLSLIITLICYHRSRNSRRNRLLQVT
TMNMTPRRPGLTRKPYQPYAPARDFAAYRPKWIRKKFPHIFKQPFKKTTGAAQEEDA
CSCRCPQEEEGGGGGYELRAKFSRSAETAANLQDPNQLYNELNLGRREEYDVLEKK
RARDPEMGGKQQRRRNPQEGVYNALQKDKMAEAYSEIGTKGERRRGKGHDGLYQ
GLSTATKDTYDALHMQTLAPRVDAAAT
Exemplary GD2 CAR with CD28 component (SEQ ID NO: 3):
MVLLVTSLLLCELPHPQVQLKESGPVLVAPSQTLSITCTVSGFSLASYNIHWVRQPPG
KGLEWLGVIWAGGSTNYNSALMSRLSISKDNSKSQVFLQMNSLQTDDTAMYYCAK
RSDDYSWFAYWGQGTLVTVSASGGGGSGGGGSGGGGSENVLTQSPAIMSASPGEKV
TMTCRASSSVSSSYLHWYQQKSGASPKVWIYSTSNLASGVPGRFSGSGSGTSYSLTIS
SVEAEDAATYYCQQYSGYPITFGAGTKVEVKRAAATTTKPVLRTPSPVHPTGTSQPQ
RPEDCRPRGSVKGTGLDFACDIYIWAPLAGICVALLLSLIITLICYHRSRNSRRNRLLQ
VTTMNMTPRRPGLTRKPYQPYAPARDFAAYRPRAKFSRSAETAANLQDPNQLYNELN
LGRREEYDVLEKKRARDPEMGGKQQRRRNPQEGVYNALQKDKMAEAYSEIGTKGE
RRRGKGHDGLYQGLSTATKDTYDALHMQTLAPR
Exemplary GD2 CAR with 4-1BB component (SEQ ID NO: 4):
MVLLVTSLLLCELPHPQVQLKESGPVLVAPSQTLSITCTVSGFSLASYNIHWVRQPPG
KGLEWLGVIWAGGSTNYNSALMSRLSISKDNSKSQVFLQMNSLQTDDTAMYYCAK
RSDDYSWFAYWGQGTLVTVSASGGGGSGGGGSGGGGSENVLTQSPAIMSASPGEKV
TMTCRASSSVSSSYLHWYQQKSGASPKVWIYSTSNLASGVPGRFSGSGSGTSYSLTIS
SVEAEDAATYYCQQYSGYPITFGAGTKVEVKRAAATTTKPVLRTPSPVHPTGTSQPQ
RPEDCRPRGSVKGTGLDFACDIYIWAPLAGICVALLLSLIITLICYHRSRKWIRKKFPHI
FKQPFKKTTGAAQEEDACSCRCPQEEEGGGGGYELRAKFSRSAETAANLQDPNQLY
NELNLGRREEYDVLEKKRARDPEMGGKQQRRRNPQEGVYNALQKDKMAEAYSEIG
TKGERRRGKGHDGLYQGLSTATKDTYDALHMQTLAPR
Exemplary CD19 CAR (SEQ ID NO: 5):
PDGTVKLLIYHTSRLHSGVPSRFSGSGSGTDYSLTISNLEQEDIATYFCQQGNTLPYTF
GGGTKLEITGGGGSGGGGSGGGGSEVKLQESGPGLVAPSQSLSVTCTVSGVSLPDYG
VSWIRQPPRKGLEWLGVIWGSETTYYNSALKSRLTIIKDNSKSQVFLKMNSLQTDDT
AIYYCAHYYYGGSYAMDYWGQGTSVTVSSAAATTTKPVLRTPSPVHPTGTSQPQRP
EDCRPRGSVKGTGLDFACDIYIWAPLAGICVALLLSLIITLICYHRSRNSRRNRLLQVT
TMNMTPRRPGLTRKPYQPYAPARDFAAYRPKWIRKKFPHIFKQPFKKTTGAAQEEDA
CSCRCPQEEEGGGGGYELRAKFSRSAETAANLQDPNQLYNELNLGRREEYDVLEKK
RARDPEMGGKQQRRRNPQEGVYNALQKDKMAEAYSEIGTKGERRRGKGHDGLYQ
GLSTATKDTYDALHMQTLAPR

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a human having cancer, wherein said method comprises administering a population of human allogenic antigen presenting cells (APCs) to said human, wherein said allogenic APCs (i) comprise nucleic acid encoding a viral vector comprising a nucleic acid sequence encoding a chimeric antigen receptor (CAR) targeting a cancer antigen of said cancer and (ii) release a population of said viral vectors within said human, wherein viral vectors of said population of released viral vectors infect a population of T cells within said human and are replication-defective within said infected T cells, wherein said infected T cells express said CAR, wherein at least some of said infected T cells expressing said CAR form dual specific effector T cells comprising said CAR, and wherein said effector T cells reduce the number of cancer cells within said human in the absence of any subsequence administration of human allogenic APCs to said human.

2. (canceled)

3. The method of claim 1, wherein said population of allogenic APCs comprises allogenic dendritic cells.

4-7. (canceled)

8. The method of claim 1, wherein said method does not comprise administering a subsequent administration of human allogenic APCs to said human.

9. A method for treating a mammal having cancer, wherein said method comprises administering a population of allogenic APCs to said mammal, wherein said allogenic APCs (i) comprise nucleic acid encoding a viral vector comprising a nucleic acid sequence encoding a CAR targeting a cancer antigen of said cancer and (ii) release a population of said viral vectors within said mammal, wherein viral vectors of said population of released viral vectors infect a population of T cells within said mammal and are replication-defective within said infected T cells, wherein said infected T cells express said CAR, wherein at least some of said infected T cells expressing said CAR form dual specific effector T cells comprising said CAR, and wherein said effector T cells reduce the number of cancer cells within said mammal.

10. The method of claim 9, wherein said mammal is a human.

11. (canceled)

12. The method of claim 1, wherein said population of allogenic APCs comprises allogenic dendritic cells.

13-15. (canceled)

16. The method of claim 9, wherein said method comprises administering a subsequent administration of allogenic APCs to said mammal.

17. The method of claim 16, wherein said subsequent administration of allogenic APCs comprises administering only allogenic APCs that lack nucleic acid encoding said viral vector.

18. The method of claim 16, wherein said subsequent administration of allogenic APCs comprises administering allogenic APCs comprising nucleic acid encoding said viral vector.

19-21. (canceled)

22. A method for generating memory T cells expressing a CAR within a human, wherein said method comprises administering a population of human allogenic APCs to said human, wherein said allogenic APCs (i) comprise nucleic acid encoding a viral vector comprising a nucleic acid sequence encoding said CAR and (ii) release a population of said viral vectors within said human, wherein viral vectors of said population of released viral vectors infect a population of T cells within said human and are replication-defective within said infected T cells, wherein said infected T cells express said CAR, and wherein at least some of said infected T cells expressing said CAR form a dual specific memory T cell within said human in the absence of a subsequent administration of human allogenic APCs to said human.

23. The method of claim 22, wherein said population of said allogenic APCs comprises allogenic dendritic cells.

24. The method of claim 22, wherein said administering step is an intravenous administration.

25. The method of claim 22, wherein said CAR targets a cancer antigen.

26. (canceled)

27. The method of claim 22, wherein said dual specific memory T cell is CD69⁺ and CD103⁺.

28. (canceled)

29. The method of claim 22, wherein said human has cancer.

30-34. (canceled)

35. A method for generating memory T cells expressing a CAR within a mammal, wherein said method comprises administering a population of APCs to said mammal, wherein said APCs (i) comprise nucleic acid encoding a viral vector comprising a nucleic acid sequence encoding said CAR and (ii) release a population of said viral vectors within said mammal, wherein viral vectors of said population of released viral vectors infect a population of T cells within said mammal and are replication-defective within said infected T cells, wherein said infected T cells express said CAR, and wherein at least some of said infected T cells expressing said CAR form a dual specific memory T cell within said mammal.

36. The method of claim 35, wherein said mammal is a human.

37. The method of claim 35, wherein said population of said allogenic APCs comprises allogenic dendritic cells.

38. The method of claim 35, wherein said administering step is an intravenous administration.

39. The method of claim 35, wherein said CAR targets a cancer antigen.

40-48. (canceled)


SEQUENCE LISTING (5 sequences)

SEQ ID NO: 1 - protein, 8 aa, synthetic construct
SIINFEKL

SEQ ID NO: 2 - protein, 542 aa, synthetic construct
MVLLVTSLLLCELPHPAFLLIPDIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKRTGSTSGSGKPGSGEGSEVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSAAATTTKPVLRTPSPVHPTGTSQPQRPEDCRPRGSVKGTGLDFACDIYIWAPLAGICVALLLSLIITLICYHRSRNSRRNRLLQVTTMNMTPRRPGLTRKPYQPYAPARDFAAYRPKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRVDAAAT

SEQ ID NO: 3 - protein, 488 aa, synthetic construct
MVLLVTSLLLCELPHPQVQLKESGPVLVAPSQTLSITCTVSGFSLASYNIHWVRQPPGKGLEWLGVIWAGGSTNYNSALMSRLSISKDNSKSQVFLQMNSLQTDDTAMYYCAKRSDDYSWFAYWGQGTLVTVSASGGGGSGGGGSGGGGSENVLTQSPAIMSASPGEKVTMTCRASSSVSSSYLHWYQQKSGASPKVWIYSTSNLASGVPGRFSGSGSGTSYSLTISSVEAEDAATYYCQQYSGYPITFGAGTKVEVKRAAATTTKPVLRTPSPVHPTGTSQPQRPEDCRPRGSVKGTGLDFACDIYIWAPLAGICVALLLSLIITLICYHRSRNSRRNRLLQVTTMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 4 - protein, 492 aa, synthetic construct
MVLLVTSLLLCELPHPQVQLKESGPVLVAPSQTLSITCTVSGFSLASYNIHWVRQPPGKGLEWLGVIWAGGSTNYNSALMSRLSISKDNSKSQVFLQMNSLQTDDTAMYYCAKRSDDYSWFAYWGQGTLVTVSASGGGGSGGGGSGGGGSENVLTQSPAIMSASPGEKVTMTCRASSSVSSSYLHWYQQKSGASPKVWIYSTSNLASGVPGRFSGSGSGTSYSLTISSVEAEDAATYYCQQYSGYPITFGAGTKVEVKRAAATTTKPVLRTPSPVHPTGTSQPQRPEDCRPRGSVKGTGLDFACDIYIWAPLAGICVALLLSLIITLICYHRSRKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 5 - protein, 476 aa, synthetic construct
PDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAHYYYGGSYAMDYWGQGTSVTVSSAAATTTKPVLRTPSPVHPTGTSQPQRPEDCRPRGSVKGTGLDFACDIYIWAPLAGICVALLLSLIITLICYHRSRNSRRNRLLQVTTMNMTPRRPGLTRKPYQPYAPARDFAAYRPKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR